(19) United States
(12) Patent Application Publication
(10) Pub. No.: US 2021/0130438 A1
(43) Pub. Date: May 6, 2021

(54) PAN-CANCER T CELL EXHAUSTION GENES

(71) Applicants: The Broad Institute, Inc., Cambridge, MA (US); Massachusetts Institute of Technology, Cambridge, MA (US); The Brigham and Women's Hospital, Inc., Boston, MA (US)

(72) Inventors: Livnat Jerby-Arnon, Cambridge, MA (US); Ana Carrizosa Anderson, Boston, MA (US); Aviv Regev, Cambridge, MA (US); Katherine Tooley, Boston, MA (US); Vijay K. Kuchroo, Boston, MA (US)

(21) Appl. No.: 17/083,235

(22) Filed: Oct. 28, 2020

Related U.S. Application Data
(60) Provisional application No. 62/927,077, filed on Oct. 28, 2019.

(51) Int. Cl. C07K 14/715; C12N 15/90; C12N 5/0783; A61K 35/17; C07K 16/28; A61K 39/395; C12Q 1/6886; G01N 33/50; A61P 35/00

(52) U.S. Cl. CPC C07K 14/7158; C12N 2510/00; C12N 5/0636; A61K 35/17; C07K 16/2866; A61K 39/3955; C07K 16/2827; C12Q 1/6886; G01N 33/505; A61P 35/00; C07K 2317/31; C12Q 2600/158; C12N 2800/80; C12N 2310/20; C12N 15/907

(57) ABSTRACT

The present invention provides novel pan-cancer T cell exhaustion regulators. CXCR6 expressed in CD8+ T cells was specifically identified as regulating anti-tumor immunity. Modulating CXCR6-CXCL16 interaction is useful in modulating anti-tumor immunity. The identified genes may be modulated in T cells for use in adoptive cell transfer. The identified genes may be modulated in vivo.

### CROSS-REFERENCE TO RELATED APPLICATIONS

This application claims the benefit of U.S. Provisional Application No. 62/927,077, filed Oct. 28, 2019. The entire contents of the above-identified application are hereby fully incorporated herein by reference.

### REFERENCE TO AN ELECTRONIC SEQUENCE LISTING

The contents of the electronic sequence listing (“BROD-4650US_ST25.txt”; Size is 10 Kilobytes and it was created on Oct. 26, 2020) is herein incorporated by reference in its entirety.

### TECHNICAL FIELD

The subject matter disclosed herein is generally directed to novel pan-cancer exhaustion regulators for use in enhancing anti-tumor immunity and detecting immune states.

### BACKGROUND

Reversing dysfunctional T cell states that arise in cancer and chronic viral infections is the focus of many immunotherapeutic interventions. Targeting T cell dysfunction is challenging, as dysfunction is closely intertwined with T cell activation (I. Tirosh et al., Dissecting the multicellular ecosystem of metastatic melanoma by single-cell RNA-seq. Science. 352, 189-196 (2016); and M. Singer et al., A Distinct Gene Module for Dysfunction Uncoupled from Activation in Tumor-Infiltrating T Cells. Cell. 166, 1500-1511.e9 (2016)), and an effective treatment often needs to reverse the T cell phenotype in a variety of tissues and microenvironments.

Citation or identification of any document in this application is not an admission that such a document is available as prior art to the present invention.

### SUMMARY

In one aspect, the present invention provides for a population of CD8+ T cells modulated ex vivo to increase expression, activity and/or function of CXCR6. In certain embodiments, a nucleotide sequence encoding for CXCR6 is introduced to the one or more CD8+ T cells ex vivo. In certain embodiments, a sequence specific genome editing system is introduced ex vivo to activate or enhance expression of endogenous CXCR6, such as by modifying the CXCR6 gene, negative regulators of CXCR6, chromatin surrounding the CXCR6 gene, the promoter or enhancers regulating the CXCR6 gene, or by substituting the CXCR6 gene with an enhanced expression cassette. In certain embodiments, the population is obtained by enriching for CXCR6+CD8+ T cells from an ex vivo population of immune cells. In certain embodiments, the T cells are further enriched for PD1+ TIM3− CD8+ T cells, whereby the population of cells is enriched for CXCR6+ PD1+ TIM3− CD8+ T cells. In certain embodiments, the T cells are enriched using antibodies specific to CXCR6, PD1, TIM3 and/or CD8. In certain embodiments, the CD8+ T cells are further modulated to comprise decreased expression, activity and/or function of one or more genes selected from the group consisting of HAVCR2, PDCD1, TIGIT, CTLA4, LAG3 and ENTPD1. In certain embodiments, the CD8+ T cells are tumor infiltrating lymphocytes (TILs). In certain embodiments, the CD8+ T cells are specific for a tumor antigen. In certain embodiments, the CD8+ T cells are modulated to express an exogenous T cell receptor (TCR) or chimeric antigen receptor (CAR). In certain embodiments, the CD8+ T cells express a suicide switch gene. In certain embodiments, the CD8+ T cells are autologous cells obtained from a subject suffering from cancer. In certain embodiments, the CD8+ T cells are allogenic cells further modulated to prevent transplant rejection.

In another aspect, the present invention provides for a pharmaceutical composition comprising the population of cells according to any embodiment herein. In another aspect, the present invention provides for a method of treating cancer comprising administering the pharmaceutical composition to a subject in need thereof.

In another aspect, the present invention provides for a method of treating cancer comprising administering to a subject in need thereof one or more agents capable of modulating expression, activity, and/or function (i.e., increasing or reducing) of CXCR6. In certain embodiments, CXCR6 expression, activity, and/or function in T cells is enhanced. In certain embodiments, CXCL16 expression, activity, and/or function is enhanced. In certain embodiments, CXCR6 expression, activity, and/or function is reduced. In certain embodiments, one or more agents capable of reducing expression, activity, and/or function of CXCR6 is administered in combination with anti-PD-1, anti-CTLA4, anti-PD-L1, anti-TIM3, anti-TIGIT, anti-LAG3, or combination thereof. In certain embodiments, the method further comprises administering one or more agents capable of decreasing expression, activity, and/or function of one or more genes selected from the group consisting of HAVCR2, PDCD1, TIGIT, CTLA4, LAG3, ENTPD1 and PD-L1. In certain embodiments, the one or more agents target a ligand, receptor or substrate of the one or more genes.

In certain embodiments, the one or more agents comprise an antibody, antibody-like protein scaffold, aptamer, small molecule, genetic modifying agent, CXCL16 protein or fragment, nucleic acid or any combination thereof. In certain embodiments, the one or more agents comprise one or more antibodies targeting CXCR6. In certain embodiments, CXCL16 is targeted by the one or more agents. In certain embodiments, the one or more agents comprise one or more antibodies targeting one or more genes selected from the group consisting of HAVCR2, PDCD1, TIGIT, CTLA4, LAG3, ENTPD1 and PD-L1. In certain embodiments, the one or more antibodies is selected from the group consisting of Ipilimumab, Nivolumab, Pembrolizumab and Atezolizumab. In certain embodiments, the one or more agents comprise an inhibitor of ENTPD1. In certain embodiments, the inhibitor is selected from the group consisting of 6-N,N-Diethyl-d-β-γ-dibromomethylene adenosine triphosphate (ARL 67156), 8-thiobutyladenosine 5′-triphosphate (8-Bu-S-ATP), polyoxymetate-1 (POM-1) and α,β-methylene ADP (APCP). In certain embodiments, the small molecule is a small molecule degrader. In certain embodiments, the genetic modifying agent comprises a CRISPR system, RNAi system, a zinc finger nuclease system, a TALE system, or a meganuclease designed to target the CXCR6 gene, target negative regulators of CXCR6, modify chromatin surrounding the CXCR6 gene, target the promoter or enhancers regulating the CXCR6 gene, or substitute the CXCR6 gene with an enhanced expression cassette.

In another aspect, the present invention provides for a method of detecting dysfunctional T cells comprising detecting a dysfunctional gene signature in T cells obtained from a subject in need thereof, wherein the dysfunctional gene signature comprises expression of: one or more genes selected from the group consisting of CXCR6, NDFIP2, CD82, LSP1, FKBP1A, PKM, ACP5, PHLDA1, AKAP5, NAB1, SIRPG, DUSP4, RGS1, GAPDH, RBPJ, TNFRSF9, MIR155HG, CD27, CD2, TNFSF4, CXCL13, SAMSN1, EPSTI1, SARDH, CD74, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1; or one or more genes selected from the group consisting of CD82, PKM, ACP5, AKAP5, NAB1, SIRPG, RGS1, TNFRSF9, MIR155HG, CD27, CD2, TNFSF4, CXCL13, SAMSN1, EPSTI1, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1; or one or more genes selected from the group consisting of CD82, PKM, ACP5, AKAP5, NAB1, SIRPG, RGS1, TNFRSF9, MIR155HG, CD27, CD2, TNFSF4, CXCL13, SAMSN1, EPSTI1, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1, and one or more genes selected from the group consisting of NDFIP2, LSP1, CXCR6, FKBP1A, PHLDA1, DUSP4, GAPDH, RBPJ, SARDH and CD74; or one or more genes selected from the group consisting of RBPJ, NAB1, TOX, IFI6, ZBED2, IFI16, CCND2, PHLDA1 and ETV1; or one or more genes selected from the group consisting of CXCR6, TNFRSF9, SIRPG, CD27, CD2, TNFSF4, HLA-DRA, CD8A, HLA-DRB1, HLA-DMA, HLA-DPA1, CD74, TRAF5, BST2, VCAM1, ITGAE, CLEC2D, CD38, ANXA5, CD82, HLA-A, TNFRSF1B, FASLG, PAG1, RAB27A, LY6E, IGFLR1, CD3D and HLA-DRB5; or one or more genes selected from the group consisting of ACP5, CXCL13, FAM3C and ISG15. In certain embodiments, the dysfunctional gene signature further comprises expression of one or more genes selected from the group consisting of HAVCR2, PDCD1, TIGIT, CTLA4, LAG3 and ENTPD1.

In certain embodiments, the method further comprises determining if the subject is responsive to checkpoint blockade (CPB) monotherapy, wherein detecting the dysfunctional gene signature in a subject indicates that the subject is not responsive to checkpoint blockade (CPB) monotherapy. In certain embodiments, the subject that is not responsive has a higher proportion of T cells expressing the dysfunctional signature as compared to T cells not expressing the dysfunctional signature.

In certain embodiments, the method further comprises treating a subject not having a dysfunctional gene signature with checkpoint blockade (CPB) monotherapy; or treating a subject having a dysfunctional signature according to any embodiment herein; or treating a subject having a dysfunctional signature with one or more treatments selected from the group consisting of surgery, targeted therapy, chemotherapy and radiation therapy; and, optionally, immunotherapy.

In certain embodiments, the method is for monitoring checkpoint blockade (CPB) therapy in a subject in need thereof, wherein the CPB therapy is effective if CXCR6 expression increases in CD8+ T cells in the subject.

In another aspect, the present invention provides for a method of screening for T cell modulating agents, comprising: treating a population of T cells having a dysfunctional gene signature according to any embodiment herein with a test agent; and detecting a decrease in the dysfunctional gene signature as compared to an untreated population of T cells.

In another aspect, the present invention provides for a kit comprising reagents to detect at least one gene according to the gene signature as defined in any embodiment herein.

In another aspect, the present invention provides for a method of identifying a pan-tumor signature comprising: applying dimensionality reduction on two or more single cell RNA sequencing cohorts comprising dysfunctional T cells simultaneously; identifying genes that characterize both dysfunctional CD8 T cells and regulatory (CD4) T cells; and using RNA velocity to identify genes that are expressed early and/or late during exhaustion. In certain embodiments, dimensionality reduction comprises mixed-NMF.

In another aspect, the present invention provides for a bispecific antibody capable of enhancing interaction between dendritic cells (DCs) and PD1+ Tim3− CD8+ T cells, wherein the bispecific antibody binds to a surface protein on the T cells and a DC surface protein. In certain embodiments, the T cell surface protein is selected from the group consisting of CXCR6 and PD1. In certain embodiments, the DC surface protein is selected from the group consisting of CXCL16, CD11c, XCR1 and CD103. In another aspect, the present invention provides for a method of treating cancer comprising administering to a subject in need thereof the bispecific antibody according to any embodiment herein.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1D—CXCR6 expression in T cells. FIG. 1A. CXCR6 mRNA expression in PD1−, Tim3− CD8+ (double negative, DN), PD1+, Tim3− CD8+(single positive, SP), and PD1+, Tim3+ CD8+(double positive, DP) T cells. FIG. 1B. CXCR6 expression by flow cytometry in PD1−, Tim3− CD8+, PD1+, Tim3− CD8+, and PD1+, Tim3+ CD8+ tumor infiltrating lymphocytes (TILs). FIG. 1C. Graph of CXCR6 expression by flow cytometry in PD1−, Tim3− CD8+, PD1+, Tim3− CD8+, and PD1+, Tim3+ CD8+ tumor infiltrating lymphocytes (TILs). FIG. 1D. FACS plots showing CXCR6 expression by in PD1−, Tim3− CD8+, PD1+, Tim3− CD8+, and PD1+, Tim3+ CD8+ tumor infiltrating lymphocytes (TILs) (left panels) and CD39 expression in CXCR6 positive cells (right panels).

FIG. 2A-2B—CXCR6 expression in melanoma T cell clusters and CXCL16 expression in melanoma non-T cell clusters. FIG. 2A. UMAP plot derived from scRNA-sequencing performed using the 10× platform on CD45+ cells sorted from B16F10 tumors. Left panel indicates T cell clusters and annotated cell types for each cluster. Right panel indicates CXCR6 expression projected onto the plot. FIG. 2B. UMAP plot derived from scRNA-sequencing performed using the 10× platform on CD45+ cells sorted from B16F10 tumors. Left panel indicates non-T cell clusters and annotated cell types for each cluster. Right panel indicates CXCL16 expression projected onto the plot.

FIG. 3A-3F—T Cell CRISPR/Cas9 KO transfer with pmel-1/B16F10 melanoma model. FIG. 3A. Diagram showing experimental design. FIG. 3B. Diagram showing experimental design. FIG. 3C. Graphs showing validation of the experimental system. (left panel) NGFR positive cells in untransduced and transduced cells. (right panel) Percentage of central memory CD62L+ (CM) and CD62L− effector memory (EM) cells in untransduced and transduced cells. FIG. 3D. Graph showing tumor size post injection in control mice and mice transferred pmel-1 CD8+ T cells. FIG. 3E. Graph showing percentage of pmel-1 CD8+ T cells in tumors from control mice and mice transferred pmel-1 CD8+ T cells. FIG. 3F. Graph showing percentage of PD1−, Tim3− CD8+, PD1+, Tim3− CD8+, and PD1+, Tim3+ CD8+ T cells in tumors from control mice and mice transferred pmel-1 CD8+ T cells.

FIG. 4A-4B—CXCR6 sgRNA CRISPR Editing. FIG. 4A. CXCR6 guide target sequence. FIG. 4B. Indel spectrum and aberrant sequence signal for CXCR6 sgRNA_2.

FIG. 5A-5B—CXCR6 expression pattern is conserved across different tumor models. FIG. 5A. Schematic showing transition of naïve T cells to dysfunctional T cells and the correlation to PD1 and TIM3 expression. FIG. 5B. CXCR6 expression in PD1−, Tim3− CD8+, PD1+, Tim3− CD8+, and PD1+, Tim3+ CD8+ tumor infiltrating lymphocytes (TILs) across three mouse tumor models.

FIG. 6—CXCR6 expression level is highest in PD1+ Tim3+ CD8+ TILs. CXCR6 expression by FACS in PD1−, Tim3− CD8+, PD1+, Tim3− CD8+, and PD1+, Tim3+ CD8+ tumor infiltrating lymphocytes (TILs) across three mouse tumor models.

FIG. 7—CXCR6+ cells express multiple inhibitory receptors. Expression by FACS of Tox, Tigit, Lag3 and CD39 in CXCR6− and CXCR6+ T cells obtained from the B16 mouse tumor model.

FIG. 8—CXCL16 is expressed in other myeloid cells and is mostly intracellular. Flow cytometric analysis of CXCL16 on B16Ova tumors.

FIG. 9—CXCL16 is expressed in all DC subsets. Flow cytometric analysis of CXCL16 on B16Ova tumors.

FIG. 10—Experimental scheme to study gene deletion in CD8⁺ T cells in vivo.

FIG. 11A-11B—CXCR6 KO CD8+ T cells fail to control tumor growth using the B16Ova/OTI T cell system. FIG. 11A. Graph showing tumor area across time points with no transfer of T cells, control transfer and transfer of CXCR6 −/− T cells. FIG. 11B. Tumor area and tumor weight at day 13 with no transfer of T cells, control transfer and transfer of CXCR6 −/− T cells. Statistics from 2-way anova with multiple comparisons: # denotes statistical significance between control and CXCR6 KO; * denotes significance between No transfer and control.

FIG. 12—Lack of CXCR6 does not affect T cell infiltration into the tumor. Graphs showing total T cell infiltration and OTI T cell infiltration after indicated transfer.

FIG. 13A-13B—CRISPR KO Cells show efficient CXCR6 Deletion in vivo. FIG. 13A. Graph showing percentage of CXCR6+ T cells in transduced and untransduced cells. FIG. 13B. FACS showing percentage of CXCR6+ T cells in transduced and untransduced cells.

FIG. 14A-14B—CXCR6 KO does not affect PD1 and Tim3 populations or CD39 expression. FIG. 14A. Graph showing percentage of PD1−, Tim3− CD8+, PD1+, Tim3− CD8+, and PD1+, Tim3+ CD8+ T cells in transduced cells. FIG. 14B. Graph showing percentage of CD39+ T cells in transduced cells.

FIG. 15A-15B—CXCR6 KO does not affect PD1 and Tim3 populations or CD39 expression. FIG. 15A. Schematic showing transition of naïve T cells to dysfunctional T cells and the correlation to TCF1 and CX3CR1 expression. FIG. 15B. Graphs showing percentage of TCF1+ and CX3CR1+ T cells in transduced cells.

FIG. 16—Differences in cytokines not observed, but an increase of Granzyme B+ cells that do not degranulate is observed. (left) Graph showing percentage of indicated cytokine+T cells in transduced cells. (right) Graph showing percentage of GrzmB+ cells in transduced cells. (Re-stimulated with 5 ug/mL SIINFEKL)

FIG. 17—Less effector differentiation in endogenous CD8+ T cells in mice receiving CXCR6-KO CD8+ T cells. (left) Graph showing percentage of PD1−, Tim3− CD8+, PD1+, Tim3− CD8+, and PD1+, Tim3+ CD8+ T cells in mice after transfer of transduced cells. (right) Graph showing percentage of CX3CR1+ T cells in mice after transfer of transduced cells.

FIG. 18—Control and KO T cell infiltration in the tumor-draining lymph node. Graph showing the number of transduced cells in the tumor draining and non-draining lymph nodes.

FIG. 19—CXCR6 expression with immune-checkpoint blockade treatment (ICB). (left) Graph showing tumor growth in mice treated and untreated with ICB. (right) Graph showing tumor growth in individual mice treated and untreated with ICB at the indicated days post tumor injection. ICB—200 ug anti-PD-L1, 200 ug anti-Tim3 in 200 uL PBS. Isotype—2A3 isotype control, 200 ug in 200 uL PBS.

FIG. 20A-20B—PD1+ Tim3− CD8+ T cells expand upon ICB and have increased CXCR6 expression. FIG. 20A. Graph showing percentage of PD1−, Tim3− CD8+, PD1+, Tim3− CD8+, and PD1+, Tim3+ CD8+ T cells after ICB or isotype control treatment. FIG. 20B. Graphs showing percentage of CXCR6+ and CXCR6− PD1−, Tim3− CD8+, PD1+, Tim3− CD8+, and PD1+, Tim3+ CD8+ T cells after ICB or isotype control treatment.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to International Patent Publication Nos. WO 2018/183921, WO 2019/070755 and WO 2018/049025. All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide a dysfunction gene module that includes novel markers. The markers include cell surface proteins, secreted proteins, transcription factors, and enzymes. The markers provide diagnostic, therapeutic and screening applications. Embodiments disclosed herein also provide T cells that are resistant to dysfunction/exhaustion. In one aspect, CXCR6 is expressed in PD1+ TIM3− CD8+ and PD1+ TIM3+ CD8+ T cells, which are T cells having intermediate and high dysfunction level along a trajectory to fully dysfuntional T cells. Applicants identified that CXCR6 expression in the T cells preserves a level of functionality in tumor-specific CD8+ T cells and that without it, T cells become even more dysfunctional or exhausted. In one embodiment, T cells are provided that are modulated to enhance expression of CXCR6. These T cells can be used in adoptive cell transfer to enhance anti-tumor immunity. In other embodiments, inhibitory exhaustion markers are reduced or knocked out in T cells. These T cells can be used in adoptive cell transfer to enhance anti-tumor immunity. In other embodiments, T cells for adoptive cell transfer can be enriched from a population of immune cells (e.g., obtained from a donor or subject in need thereof). For example, CXCR6+ T cells can be enriched and/or exhausted T cells can be depleted using the exhaustion markers identified. Applicants also identified that checkpoint blockade therapy increases the expression of CXCR6+ in PD1+ TIM3− CD8+ T cells and this correlates to anti-tumor immunity in mouse tumor models. These cells can be enriched and used for adoptive cell transfer. Thus, embodiments disclosed herein provide methods of enhancing anti-tumor immunity using the T cells modulated or enriched for enhance CXCR6 expression or activity.

Embodiments disclosed herein also provide methods of reversing and/or blocking T cell exhaustion/dysfunction, methods of detecting exhausted T cells, and screening for agents capable of modulating T cell exhaustion. For example, surface proteins, secreted proteins, transcription factors, and enzymes expressed in exhausted T cells can be targeted to prevent suppression of anti-tumor immunity by the exhausted T cells (e.g., CXCR6). Moreover, T cell exhaustion can be reversed by binding to specific biomarkers associated with the exhausted T cells. Applicants identified that CXCR6+ T cells interact with CXCL16 expressing myeloid cells and that the interaction is associated with anti-tumor immunity. Applicants provide for bispecific antibodies capable of increasing the interaction by binding to surface proteins on the cells. Bispecific antibodies can be generated using any known binding proteins (e.g., antibodies) specific for the surface markers disclosed. Applicants also identified that CXCR6 knockout increases TCF-1 and decreases CX3CR1 in T cells. These T cells may respond to checkpoint blockade therapy to enhance anti-tumor immunity and a combination treatment reducing CXCR6 and administering checkpoint blockade therapy can increase anti-tumor immunity.

To gain a deeper molecular understanding of T cell dysfunction, Applicants analyzed the transcriptomes of 51,935 T cells, collected from more than a hundred cancer patients (L. Jerby-Arnon et al., A Cancer Cell Program Promotes T Cell Exclusion and Resistance to Checkpoint Blockade. Cell. 175, 984-997.e24 (2018); C. Zheng et al., Landscape of Infiltrating T Cells in Liver Cancer Revealed by Single-Cell Sequencing. Cell. 169, 1342-1356.e16 (2017); L. Zhang et al., Lineage tracking reveals dynamic relationships of T cells in colorectal cancer. Nature. 564, 268-272 (2018); D. Lambrechts et al., Phenotype molding of stromal cells in the lung tumor microenvironment. Nat. Med. 24, 1277-1289 (2018); M. Sade-Feldman et al., Defining T Cell States Associated with Response to Checkpoint Immunotherapy in Melanoma. Cell. 175, 998-1013.e20 (2018); and E. Azizi et al., Single-Cell Map of Diverse Immune Phenotypes in the Breast Tumor Microenvironment. Cell. 174, 1293-1308.e36 (2018)). The data represents 5 major tumor types: melanoma, breast, lung, colon, and liver cancer. Applicants developed a computational approach to analyze these cohorts in unison and identified a distinct gene module for T cell dysfunction. Unlike most dysfunction markers, this module is uncoupled from T cell activation. The dysfunction module generalizes across cancer types and is evolutionary conserved. The dysfunction module also marks dysfunctional I cells in mouse models. The module includes immune checkpoints and multiple genes which have been shown to promote T cell dysfunction. Analyzing scRNA-Seq profiles of T cells collected from melanoma patients prior to immune checkpoint blockade (ICB) (M. Sade-Feldman et al., Defining T Cell States Associated with Response to Checkpoint Immunotherapy in Melanoma. Cell. 175, 998-1013.e20 (2018)), Applicants show that the dysfunction module accurately predicts the subsequent clinical response and captures aspects of T cell dysfunction which are not full reversed by CTLA-4 and PD-1 blockade. The dysfunction genes in CD8 T cells may effectively reverse the dysfunction phenotype and trigger antitumor immunity. This can be tested in mouse models. Taken together, the findings and approach provide novel targets for studying and modulating dysfunctional T cell states.

### Therapeutic Methods

In certain embodiments, the present invention provides for CD8+ T cells modulated to enhance anti-tumor immunity. As used herein, “modulating”, “to modulate”, “modifying” or “to modify” generally means either reducing or inhibiting the expression or activity of, or alternatively increasing the expression or activity of a target (e.g., CXCR6). As used herein “modify” and “modulate” are used interchangeably. In particular, “modulating” or “to modulate” can mean either reducing or inhibiting the activity of, or alternatively increasing a (relevant or intended) biological activity of, a target or antigen as measured using a suitable in vitro, cellular or in vivo assay (which will usually depend on the target involved), by at least 5%, at least 10%, at least 25%, at least 50%, at least 60%, at least 70%, at least 80%, at least 90%, or more, compared to activity of the target in the same assay under the same conditions but without the presence of an agent. An “increase” or “decrease” refers to a statistically significant increase or decrease respectively. For the avoidance of doubt, an increase or decrease will be at least 10% relative to a reference, such as at least 10%, at least 20%, at least 30%, at least 40%, at least 50%, at least 60%, at least 70%, at least 80%, at least 90%, at least 95%, at least 97%, at least 98%, or more, up to and including at least 100% or more, in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more. “Modulating” can also involve effecting a change (which can either be an increase or a decrease) in affinity, avidity, specificity and/or selectivity of a target or antigen. “Modulating” can also mean effecting a change with respect to one or more biological or physiological mechanisms, effects, responses, functions, pathways or activities in which the target or antigen (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist can be determined in any suitable manner and/or using any suitable assay known or described herein (e.g., in vitro or cellular assay), depending on the target or antigen involved.

Modulating can, for example, also involve allosteric modulation of the target and/or reducing or inhibiting the binding of the target to one of its substrates or ligands and/or competing with a natural ligand, substrate for binding to the target. Modulating can also involve activating the target or the mechanism or pathway in which it is involved. Modulating can, for example, also involve effecting a change in respect of the folding or confirmation of the target, or in respect of the ability of the target to fold, to change its conformation (for example, upon binding of a ligand), to associate with other (sub)units, or to disassociate. Modulating can, for example, also involve effecting a change in the ability of the target to signal, phosphorylate, dephosphorylate, and the like.

### Adoptive Cell Transfer

In certain embodiments, CD8+ T cells positive for CXCR6 are used for adoptive cell transfer (ACT). As used herein, “ACT”, “adoptive cell therapy” and “adoptive cell transfer” may be used interchangeably. In certain embodiments, agonists of CXCR6 expression or activity are used in ACT. In certain embodiments, T cells enriched using one or more antibodies specific for CXCR6 are used for adoptive cell transfer. In certain embodiments, CXCR6+ PD1+ TIM3+ CD8+ T cells are enriched and used for adoptive cell transfer. In certain embodiments, T cells modulated to have increased expression, activity or function for CXCR6 are used for adoptive cell transfer. In certain embodiments, a nucleotide sequence encoding for CXCR6 is introduced to the one or more CD8+ T cells ex vivo (e.g., by use of a vector, such as a viral vector). In certain embodiments, a sequence specific genome editing system is introduced ex vivo to activate or enhance expression of endogenous CXCR6 (e.g., by targeting an activator or repressor to the endogenous gene, or by editing the gene to make a more active or stable protein). In certain embodiments, the T cells are further modulated to have decreased expression, activity, and/or function of one or more exhaustion regulators described herein and may be used in adoptive cell transfer. In certain embodiments, the T cells are modified and expanded. In certain embodiments, cells with the desired phenotype are selected for and expanded. In certain embodiments, the T cells are formulated into a pharmaceutical composition. The modified T cells may be resistant to exhaustion induced by a tumor or tumor microenvironment and have enhanced ant-tumor activity. In other words, a tumor may target immune cells or the tumor microenvironment to induce a dysfunctional immune state. In certain embodiments, modulating one or more identified therapeutic targets in an immune cell shifts the immune cell to be resistant to dysfunction or have increased effector function. In certain embodiments, the immune cells prevent an immune suppressive tumor microenvironment. Such immune cells may be used to increase the effectiveness of adoptive cell transfer. In certain embodiments, immune cells are modulated using a genetic modifying agent, antibody or small molecule, described further herein.

In certain embodiments, Adoptive Cell Therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive Cell Therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (Dl), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CIVIL), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CD5, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI la-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3 chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS)) (SEQ ID NO: 1). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ ID NO: 2) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO: 2) and continuing all the way to the carboxy-terminus of the protein. The sequence is reproduced herein: IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS (SEQ ID NO: 1). Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signalling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signalling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may be eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CART cells of this kind may for example be used in animal models, for example to treat tumor xenografts.

In certain embodiments, ACT includes co-transferring CD4+ Th1 cells and CD8+ CTLs to induce a synergistic antitumour response (see, e.g., Li et al., Adoptive cell therapy with CD4+ T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4⁺ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MEW restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CART cells, Sci Transl Med. 2017 Jan. 25; 9(374); Legut, et al., 2018, CRISPR-mediated TCR replacement generates superior anticancer transgenic T cells. Blood, 131(3), 311-322; Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018; and Roth, T. L. Editing of Endogenous Genes in Cellular Immunotherapies. Curr Hematol Malig Rep 15, 235-240 (2020)). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, a and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

International Patent Publication No. WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, International Patent Publication No. WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CART cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in International Patent Publication No. WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in International Patent Publication Nos. WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment, T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated and/or enriched. A preferred method is cell sorting and/or selection via positive or negative magnetic immunoadherence or flow cytometry that uses a cocktail of monoclonal antibodies directed to cell surface markers present on the cells positively and negatively selected. In certain embodiments, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. In certain embodiments, CXCR6+ CD8+ T cells are enriched using CXCR6 antibodies. In certain PD1 antibodies can be used to enrich CD8+ T cells. In certain embodiments, Tim3 antibodies can be used for negative selection. In certain embodiments, dendritic cell are enriched using surface markers specific to dendritic cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, CD8+ T cells are negatively enriched using Tim3 antibodies. For example to enrich for PD1+ Tim3− CXCR6+ T cells.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MEW tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MEW molecule of interest and any antigen of interest as described herein. Specific epitopes to be used in this context can be identified using numerous assays known in the art. For example, the ability of a polypeptide to bind to MEW class I may be evaluated indirectly by monitoring the ability to promote incorporation of ¹²⁵I labeled β2-microglobulin (β2m) into MHC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in International Patent Publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in International Patent Publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in International Patent Publication No. WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in International Patent Publication No. WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in International Patent Publication No. WO2016191756 comprising administering to the patient a dose of cyclophosphamide between 200 mg/m2/day and 2000 mg/m2/day and a dose of fludarabine between 20 mg/m2/day and 900 mg/m²/day.

In certain embodiments, a patient in need of adoptive cell transfer may be administered a TLR agonist to enhance anti-tumor immunity (see, e.g., Urban-Wojciuk, et al., The Role of TLRs in Anti-cancer Immunity and Tumor Rejection, Front Immunol. 2019; 10: 2388; and Kaczanowska et al., TLR agonists: our best frenemy in cancer immunotherapy, J Leukoc Biol. 2013 June; 93(6): 847-863). In certain embodiments, TLR agonists are delivered in a nanoparticle system (see, e.g., Buss and Bhatia, Nanoparticle delivery of immunostimulatory oligonucleotides enhances response to checkpoint inhibitor therapeutics, Proc Natl Acad Sci USA. 2020 Jun. 3; 202001569). In certain embodiments, the agonist is a TLR9 agonist. Id.

### Vectors

In certain embodiments, a polynucleotide sequence encoding for CXCR6 is introduced to T cells for use in adoptive cell transfer. Polynucleotides may be delivered via liposomes, particles (e.g. nanoparticles), exosomes, microvesicles or a gene-gun. In certain embodiments, a vector encoding for CXCR6 (or any gene) is introduced to a population of T cells for adoptive cell transfer. In any of the described methods one or more polynucleotide molecules may be comprised in a delivery system, or the one or more vectors may be comprised in a delivery system. Alternative techniques may be used to transform T cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon. In certain embodiments, inducible gene switches are used to regulate expression of CXCR6 or any other gene (e.g., CAR, TCR) (see, e.g., Chakravarti, Deboki et al. “Inducible Gene Switches with Memory in Human T Cells for Cellular Immunotherapy.” ACS synthetic biology vol. 8, 8 (2019): 1744-1754).

A vector refers to a nucleic acid molecule capable of transporting another nucleic acid to which it has been linked. A vector may be a replicon, such as a plasmid, phage, or cosmid, into which another DNA segment may be inserted so as to bring about the replication of the inserted segment. Generally, a vector is capable of replication when associated with the proper control elements. Examples of vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Vectors are capable of directing the expression of genes to which they are operatively-linked. Such vectors are referred to herein as “expression vectors.” Common expression vectors of utility in recombinant DNA techniques are often in the form of plasmids. A vector may be a recombinant expression vector that comprises a nucleic acid of the invention in a form suitable for expression of the nucleic acid in a host cell, which means that the recombinant expression vectors include one or more regulatory elements, which may be selected on the basis of the host cells to be used for expression, that is, operatively-linked to the nucleic acid sequence to be expressed. As used herein, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

A vector may be a viral vector, wherein virally-derived DNA or RNA sequences are present in the vector for packaging into a virus. Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some embodiments, vectors herein are lentiviral vectors. For example, the vectors may be packaged in lentiviruses. The vectors may be delivered into cells that are transduced by the lentiviruses. Within the cells, the vectors or portions thereof may be integrated into the genome of the cells. A lentiviral vector may be a vector derived from at least a portion of a lentivirus genome, including a self-inactivating lentiviral vector. Lentiviral vectors are a type of retrovirus that can infect both dividing and nondividing cells because their preintegration complex (virus “shell”) can get through the intact membrane of the nucleus of the target cell. Examples of lentivirus vectors that may be used in the clinic include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

In some embodiments, the vector is a adeno-associated virus (AAV) vector. The term “adeno-associated virus vector” or “AAV vector” refers to a vector comprising a viral genome based a serotype of Adeno-Associated Virus genome, and optionally additional nucleotide sequences (functional genes, transgenes, promoters, enhancers and any other desired gene sequences) that are inserted into the vector through cloning or any other method known in the art of recombinant genetic engineering, which is capable of transducing (infecting) cells and expressing these additional nucleotide sequences in the transduced cells. In some embodiments, the viral vector is a recombinant AAV (rAAV). Examples of rAAVs include rAAV6, rAAV1, rAAV7, rAAV8, and rAAV 9. In some examples, the rAAV is rAAV6. Eleven serotypes of AAV have thus far been identified, with the best characterized and most commonly used being AAV2. These serotypes differ in their tropism, or the types of cells they infect, making AAV a very useful system for preferentially transducing specific cell types.

### Therapeutic Agents

In certain embodiments, the present invention provides for one or more therapeutic agents to treat cancer. Targeting the identified exhaustion markers (e.g., CXCR6) may provide for enhanced or otherwise previously unknown activity in the treatment of disease. In certain embodiments, the agents are used to modulate cell types (e.g., shifting signatures or immune states). In certain embodiments, the one or more agents comprises a small molecule, small molecule degrader (e.g., PROTAC), genetic modifying agent, antibody, antibody fragment, antibody-like protein scaffold, aptamer, protein, or any combination thereof. The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

### CXCR6

In certain embodiments, CXCR6 agonists or antagonists are used to modulate an immune response. In certain embodiments, knockout or reduced CXCR6+ expression or activity reduces anti-tumor immunity. In certain embodiments, CXCR6 expression or activity maintains or enhances anti-tumor immunity. In certain embodiments, CXCR6 expression is important for maintaining anti-tumor immunity in ACT. In certain embodiments, CXCR6 expression, activity, and/or function is enhanced. In certain embodiments, CXCL16 expression, activity, and/or function is enhanced. In certain embodiments, the ligand for CXCR6 is administered (e.g., CXCL16 protein or fragment). In certain embodiments, the ligand is modified to be more stable in vivo.

In certain embodiments, CXCR6 expression, activity, and/or function is reduced. In certain embodiments, inhibitors of CXCR6 or CXCR6 antagonists are used to reduce CXCR6 expression, activity, and/or function. In certain embodiments, reduced CXCR6 increases TCF-1 and decreases CX3CR1 in CD8 T cells. Tcf7+ and CX3CR1− cells share features with CD8+ T cells associated with good prognosis and response to CPB therapy (see, e.g., US20200149009A1; and Kurtulus S, Madi A, Escobar G, et al. Checkpoint Blockade Immunotherapy Induces Dynamic Changes in PD-1-CD8+ Tumor-Infiltrating T Cells. Immunity. 2019; 50(1):181-194.e6). In certain embodiments, CXCR6 expression, activity, and/or function is reduced and checkpoint blockade therapy is administered before or after TCF-1 increase and CX3CR1 decrease. In certain embodiments, the inhibitor targets CXCR6. In certain embodiments, the inhibitor targets CXCL16. In certain embodiments, the inhibitor is a blocking antibody, described further herein. (see, e.g., WO2012082470A2; and U.S. Pat. No. 7,208,152B2).

As used herein CXCR6 refers to C—X—C motif chemokine receptor 6 (Also known as: BONZO, CD186, CDw186, STRL33, TYMSTR). Example sequences can be accessed using the following NCBI accession numbers: NM_006564.2, NM_001386435.1, NM_001386436.1, NM_001386437.1, NP_006555.1, NP_001373364.1, NP_001373365.1, and NP_001373366.1.

### PKM

In certain embodiments, inhibitors of PKM are used to reduce dysfunction. In certain embodiments, the inhibitor is a small molecule (see, e.g., U.S. Pat. No. 8,877,791B2).

### Standard of Care

Aspects of the invention involve modifying the therapy within a standard of care based on the detection of any of the biomarkers as described herein. In certain embodiments, the therapeutic agents are administered within the standard of care. In one embodiment, therapy comprising an agent is administered within a standard of care where addition of the agent is synergistic within the steps of the standard of care. In one embodiment, the agent targets and/or shifts a tumor to an immunotherapy responder phenotype. In one embodiment, the agent inhibits expression or activity of one or more transcription factors capable of regulating a gene program. In one embodiment, the agent targets tumor cells expressing a gene program. The term “standard of care” as used herein refers to the current treatment that is accepted by medical experts as a proper treatment for a certain type of disease and that is widely used by healthcare professionals. Standard of care is also called best practice, standard medical care, and standard therapy. Standards of care for cancer generally include surgery, lymph node removal, radiation, chemotherapy, targeted therapies, antibodies targeting the tumor, and immunotherapy. Immunotherapy can include checkpoint blockers (CBP), chimeric antigen receptors (CARs), and adoptive T-cell therapy. The standards of care for the most common cancers can be found on the website of National Cancer Institute (www.cancer.gov/cancertopics). A treatment clinical trial is a research study meant to help improve current treatments or obtain information on new treatments for patients with cancer. When clinical trials show that a new treatment is better than the standard treatment, the new treatment may be considered the new standard treatment.

The term “Adjuvant therapy” as used herein refers to any treatment given after primary therapy to increase the chance of long-term disease-free survival. The term “Neoadjuvant therapy” as used herein refers to any treatment given before primary therapy. The term “Primary therapy” as used herein refers to the main treatment used to reduce or eliminate the cancer. In certain embodiments, an agent that shifts a tumor to a responder phenotype are provided as a neoadjuvant before CPB therapy.

### Checkpoint Blockade Therapy

In certain embodiments, targeting an exhaustion marker (e.g., CXCR6) in combination with administering checkpoint blockade (CPB) therapy can enhance an immune response. In certain embodiments, CPB therapy is administered in combination with one or more agents capable of modulating one or more genes selected from CXCR6, NDFIP2, CD82, LSP1, FKBP1A, PKM, ACP5, PHLDA1, AKAP5, NAB1, SIRPG, DUSP4, RGS1, GAPDH, RBPJ, TNFRSF9, MIR155HG, CD27, CD2, TNFSF4, CXCL13, SAMSN1, EPSTI1, SARDH, CD74, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1, preferably, CXCR6, LSP1, CD82, PKM, NDFIP2, FKBP1A, and DUSP4.

Immunotherapy can include checkpoint blockers (CBP), chimeric antigen receptors (CARs), and adoptive T-cell therapy. Antibodies that block the activity of checkpoint receptors, including CTLA-4, PD-1, Tim-3, Lag-3, and TIGIT, either alone or in combination, have been associated with improved effector CD8⁺ T cell responses in multiple pre-clinical cancer models (Johnston et al., 2014. The immunoreceptor TIGIT regulates antitumor and antiviral CD8(+) T cell effector function. Cancer cell 26, 923-937; Ngiow et al., 2011. Anti-TIM3 antibody promotes T cell IFN-gamma-mediated antitumor immunity and suppresses established tumors. Cancer research 71, 3540-3551; Sakuishi et al., 2010. Targeting Tim-3 and PD-1 pathways to reverse T cell exhaustion and restore anti-tumor immunity. The Journal of experimental medicine 207, 2187-2194; and Woo et al., 2012. Immune inhibitory molecules LAG-3 and PD-1 synergistically regulate T-cell function to promote tumoral immune escape. Cancer research 72, 917-927). Similarly, blockade of CTLA-4 and PD-1 in patients (Brahmer et al., 2012. Safety and activity of anti-PD-L1 antibody in patients with advanced cancer. The New England journal of medicine 366, 2455-2465; Hodi et al., 2010. Improved survival with ipilimumab in patients with metastatic melanoma. The New England journal of medicine 363, 711-723; Schadendorf et al., 2015. Pooled Analysis of Long-Term Survival Data From Phase II and Phase III Trials of Ipilimumab in Unresectable or Metastatic Melanoma. Journal of clinical oncology: official journal of the American Society of Clinical Oncology 33, 1889-1894; Topalian et al., 2012. Safety, activity, and immune correlates of anti-PD-1 antibody in cancer. The New England journal of medicine 366, 2443-2454; and Wolchok et al., 2017. Overall Survival with Combined Nivolumab and Ipilimumab in Advanced Melanoma. The New England journal of medicine 377, 1345-1356) has shown increased frequencies of proliferating T cells, often with specificity for tumor antigens, as well as increased CD8⁺ T cell effector function (Ayers et al., 2017. IFN-gamma-related mRNA profile predicts clinical response to PD-1 blockade. The Journal of clinical investigation 127, 2930-2940; Das et al., 2015. Combination therapy with anti-CTLA-4 and anti-PD-1 leads to distinct immunologic changes in vivo. Journal of immunology 194, 950-959; Gubin et al., 2014. Checkpoint blockade cancer immunotherapy targets tumour-specific mutant antigens. Nature 515, 577-581; Huang et al., 2017. T-cell invigoration to tumour burden ratio associated with anti-PD-1 response. Nature 545, 60-65; Kamphorst et al., 2017. Proliferation of PD-1+ CD8 T cells in peripheral blood after PD-1-targeted therapy in lung cancer patients. Proceedings of the National Academy of Sciences of the United States of America 114, 4993-4998; Kvistborg et al., 2014. Anti-CTLA-4 therapy broadens the melanoma-reactive CD8+ T cell response. Science translational medicine 6, 254ra128; van Rooij et al., 2013. Tumor exome analysis reveals neoantigen-specific T-cell reactivity in an ipilimumab-responsive melanoma. Journal of clinical oncology: official journal of the American Society of Clinical Oncology 31, e439-442; and Yuan et al., 2008. CTLA-4 blockade enhances polyfunctional NY-ESO-1 specific T cell responses in metastatic melanoma patients with clinical benefit. Proceedings of the National Academy of Sciences of the United States of America 105, 20410-20415). Accordingly, the success of checkpoint receptor blockade has been attributed to the binding of blocking antibodies to checkpoint receptors expressed on dysfunctional CD8⁺ T cells and restoring effector function in these cells. The check point blockade therapy may be an inhibitor of any check point protein described herein. The checkpoint blockade therapy may comprise anti-TIM3, anti-CTLA4, anti-PD-L1, anti-PD1, anti-TIGIT, anti-LAG3, or combinations thereof. Anti-PD1 antibodies are disclosed in U.S. Pat. No. 8,735,553. Antibodies to LAG-3 are disclosed in U.S. Pat. No. 9,132,281. Anti-CTLA4 antibodies are disclosed in U.S. Pat. Nos. 9,327,014; 9,320,811; and 9,062,111. Specific check point inhibitors include, but are not limited to anti-CTLA4 antibodies (e.g., Ipilimumab and tremelimumab), anti-PD-1 antibodies (e.g., Nivolumab, Pembrolizumab), and anti-PD-L1 antibodies (e.g., Atezolizumab).

CD39 is an ectonucleotidase that plays an important role in the adenosine pathway, which in turn modulates the tumor microenvironment by reducing cytotoxicity function of effector (T and NK) cells and by increasing the abundance of suppressive cells (e.g. M2 macrophages, myeloid derived suppressor cells and regulatory T-cells) (see, e.g., Young, A., Mittal, D., Stagg, J. & Smyth, M. J. Targeting cancer-derived adenosine: new therapeutic approaches. Cancer Discov 4, 879-888, doi:10.1158/2159-8290.CD-14-0341 (2014)). As used herein, the term “CD39” has its general meaning in the art and refers to the CD39 protein also named as ectonucleoside triphosphate diphosphohydrolase-1 (ENTPD1). CD39 is an ectoenzyme that hydrolases ATP/UTP and ADP/UDP to the respective nucleosides such as AMP. Accordingly, the term “CD39 inhibitor” refers to a compound that inhibits the activity or expression of CD39. In some embodiments, the CD39 inhibitor is an antibody having specificity for CD39. In certain embodiments, the CD39 inhibitor is a small molecule. CD39 activity modulators are well known in the art. For example, 6-N,N-Diethyl-d-β-γ-dibromomethylene adenosine triphosphate (ARL 67156) (Levesque et al (2007) Br, J. Pharmacol, 152: 141-150; Crack et al. (1959) Br. J. Pharmacol. 114: 475-481; Kennedy et al. (1996) Semtn. Neurosci. 8: 195-199) and 8-thiobutyladenosine 5′-triphosphate (8-Bu-S-ATP) are small molecule CD39 inhibitors (Gendron et al. (2000) J Med Chem. 43:2239-2247). Other small molecule CD39 inhibitors, such as polyoxymetate-1 (POM-1) and α,β-methylene ADP (APCP), are also well known in the art (see, U.S.2010/204182 and US2013/0123345; U.S. Pat. No. 6,617,439). In addition, nucleic acid and antibody inhibitors of CD39 are also well known in the art (see, e.g., US20130273062A1; and Perrot et al., Blocking Antibodies Targeting the CD39/CD73 Immunosuppressive Pathway Unleash Immune Responses in Combination Cancer Therapies. Cell Rep. 2019 May 21; 27(8):2411-2425.e9. doi: 10.1016/j.celrep.2019.04.091).

### Antibodies

In certain embodiments, the one or more agents is an antibody. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG—IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Bispecific Antibodies

In certain embodiments, the one or more therapeutic agents can be bispecific antigen-binding constructs, e.g., bispecific antibodies (bsAb) or BiTEs, that bind two antigens (see, e.g., Suurs et al., A review of bispecific antibodies and antibody constructs in oncology and clinical challenges. Pharmacol Ther. 2019 September; 201:103-119; and Huehls, et al., Bispecific T cell engagers for cancer immunotherapy. Immunol Cell Biol. 2015 March; 93(3): 290-296). The bispecific antigen-binding construct includes two antigen-binding polypeptide constructs, e.g., antigen binding domains, wherein at least one polypeptide construct specifically binds to a surface protein. In some embodiments, the antigen-binding construct is derived from known antibodies or antigen-binding constructs. In some embodiments, the antigen-binding polypeptide constructs comprise two antigen binding domains that comprise antibody fragments. In some embodiments, the first antigen binding domain and second antigen binding domain each independently comprises an antibody fragment selected from the group of: an scFv, a Fab, and an Fc domain. The antibody fragments may be the same format or different formats from each other. For example, in some embodiments, the antigen-binding polypeptide constructs comprise a first antigen binding domain comprising an scFv and a second antigen binding domain comprising a Fab. In some embodiments, the antigen-binding polypeptide constructs comprise a first antigen binding domain and a second antigen binding domain, wherein both antigen binding domains comprise an scFv. In some embodiments, the first and second antigen binding domains each comprise a Fab. In some embodiments, the first and second antigen binding domains each comprise an Fc domain. Any combination of antibody formats is suitable for the bi-specific antibody constructs disclosed herein.

In certain embodiments, immune cells can be engaged to other immune cells. In certain embodiments, immune cells are engaged with a bsAb having affinity for both of the immune cells. In certain embodiments, CXCR6+ PD1+ CD8+ T cells interact with dendritic cells. The T cells may be CXCR6+ PD1+ TIM3− CD8+ T cells or CXCR6+ PD1+ TIM3+ CD8+ T cells. In certain embodiments, CPB therapy increases CXCR6+ PD1+ TIM3− CD8+ T cells and increases the interaction of this subset of T cells with dendritic cells. In certain embodiments, the interaction of CXCR6+ PD1+ TIM3+ CD8+ T cells (i.e., the most dysfunctional T cells) with dendritic cells prevents the T cells from becoming even more dysfunctional, preserving a level of functionality in tumor-specific CD8+ T cells. In certain embodiments, the CXCR6-CXCL16 interaction affects myeloid populations which in turn interact with endogenous CD8 T cells less effectively, resulting less anti-tumor immunity from all CD8 T cells. In certain embodiments, enhancing the CXCR6-CXCL16 interaction enhances anti-tumor immunity. In certain embodiments, the bi-specific antibody binds to a surface protein on CXCR6+ CD8+ T cells and on CXCL16 expressing myeloid cells. In certain embodiments, the surface proteins are expressed on CXCR6+ PD1+ TIM3− CD8+ T cells. In certain embodiments, the surface proteins are expressed on CXCR6+ PD1+ TIM3+ CD8+ T cells. In certain embodiments, the surface protein is expressed on dendritic cells (see, e.g., Durai V, Murphy K M. Functions of Murine Dendritic Cells. Immunity. 2016 Oct. 18; 45(4):719-736; Collin M, Bigley V. Human dendritic cell subsets: an update. Immunology. 2018 May; 154(1):3-20; and Villani A C, Satija R, Reynolds G, et al. Single-cell RNA-seq reveals new types of human blood dendritic cells, monocytes, and progenitors. Science. 2017; 356(6335)). In certain embodiments, the dendritic cells are migratory dendritic cells. In certain embodiments, the surface markers is selected from CXCL16, CD11c, XCR1 and CD103.

In certain embodiments, cells are targeted with a bsAb having affinity for both the cell and a payload. In certain embodiments, two targets are disrupted on a cell by the bsAb (e.g., two surface markers). By means of an example, an agent, such as a bispecific antibody, specifically binds to a gene product expressed on the cell surface of immune cells.

### Aptamers

In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-O-methyl (2′-OMe) substituents. Modifications of aptamers may also include, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colo.). In certain embodiments, the present invention may utilize any aptamer containing any modification as described herein.

### Small Molecules

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking a receptor binding site or activating a receptor by binding to a ligand binding site).

One type of small molecule applicable to the present invention is a degrader molecule (see, e.g., Ding, et al., Emerging New Concepts of Degrader Technologies, Trends Pharmacol Sci. 2020 July; 41(7):464-474). The terms “degrader” and “degrader molecule” refer to all compounds capable of specifically targeting a protein for degradation (e.g., ATTEC, AUTAC, LYTAC, or PROTAC, reviewed in Ding, et al. 2020). Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810). In certain embodiments, LYTACs are particularly advantageous for cell surface proteins as described herein (e.g., CXCR6).

### Genetic Modifying Agents

In certain embodiments, cells to be used in adoptive cell transfer (e.g., CAR T cells) are modified ex vivo by a genetic modifying agent described further below. In certain embodiments, a composition comprising a genetic modify agent is used a therapeutic agent. In certain embodiments, the one or more modulating agents may be a genetic modifying agent. The genetic modifying agents may manipulate nucleic acids (e.g., genomic DNA or mRNA). The genetic modulating agent can be used to up- or downregulate expression of a gene either by targeting a nuclease or functional domain to a DNA or RNA sequence. The genetic modifying agent may comprise a CRISPR system, a zinc finger nuclease system, a TALEN, a meganuclease or RNAi system. In certain embodiments, an activator is recruited by a genetic modifying agent to a regulatory sequence controlling expression of the CXCR6 gene (e.g., an enhancer). Enhancers are known in the art and can be identified (see, e.g., Fulco, et al. Activity-by-contact model of enhancer-promoter regulation from thousands of CRISPR perturbations. Nat Genet. 2019; 51(12):1664-1669. doi:10.1038/s41588-019-0538-0; Moonen, et al., 2020, KLF4 Recruits SWI/SNF to Increase Chromatin Accessibility and Reprogram the Endothelial Enhancer Landscape under Laminar Shear Stress. bioRxiv 2020.07.10.195768, doi.org/10.1101/2020.07.10.195768; Ernst, J. et al. Mapping and analysis of chromatin state dynamics in nine human cell types. Nature 473, 43-49 (2011); Lindblad-Toh, K. et al. A high-resolution map of human evolutionary constraint using 29 mammals. Nature 478, 476-482 (2011); Trynka, G. et al. Chromatin marks identify critical cell types for fine mapping complex trait variants. Nature Genet. 45, 124-130 (2013); Maurano, M. T. et al. Systematic localization of common disease-associated variation in regulatory DNA. Science 337, 1190-1195 (2012); Kundaje, A., Meuleman, W., Ernst, J. et al. Integrative analysis of 111 reference human epigenomes. Nature 518, 317-330 (2015); and egg2.wustl.edu/roadmap/web_portal/index.html). In certain embodiments, a genetic modifying agent is used to knock out or repress negative regulators of CXCR6 expression. In certain embodiments, a genetic modifying agent recruits a chromatin modifying protein to edit the chromatin of T cells to enhance expression of CXCR6. For example, chromatin modifications associated with gene activation (see, e.g., Handy D E, Castro R, Loscalzo J. Epigenetic modifications: basic mechanisms and role in cardiovascular disease. Circulation. 2011; 123(19):2145-2156).

### CRISPR-Cas Modification

In some embodiments, a polynucleotide of the present invention described elsewhere herein can be modified using a CRISPR-Cas and/or Cas-based system (e.g., genomic DNA or mRNA, preferably, for a disease gene). The nucleotide sequence may be or encode one or more components of a CRISPR-Cas system. For example, the nucleotide sequences may be or encode guide RNAs. The nucleotide sequences may also encode CRISPR proteins, variants thereof, or fragments thereof.

In general, a CRISPR-Cas or CRISPR system as used herein and in other documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two classes are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

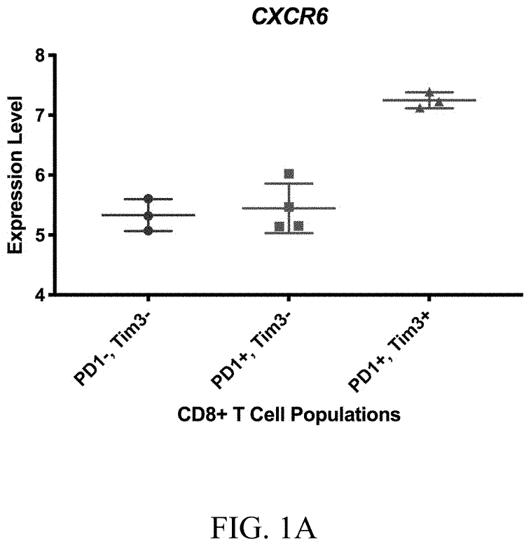

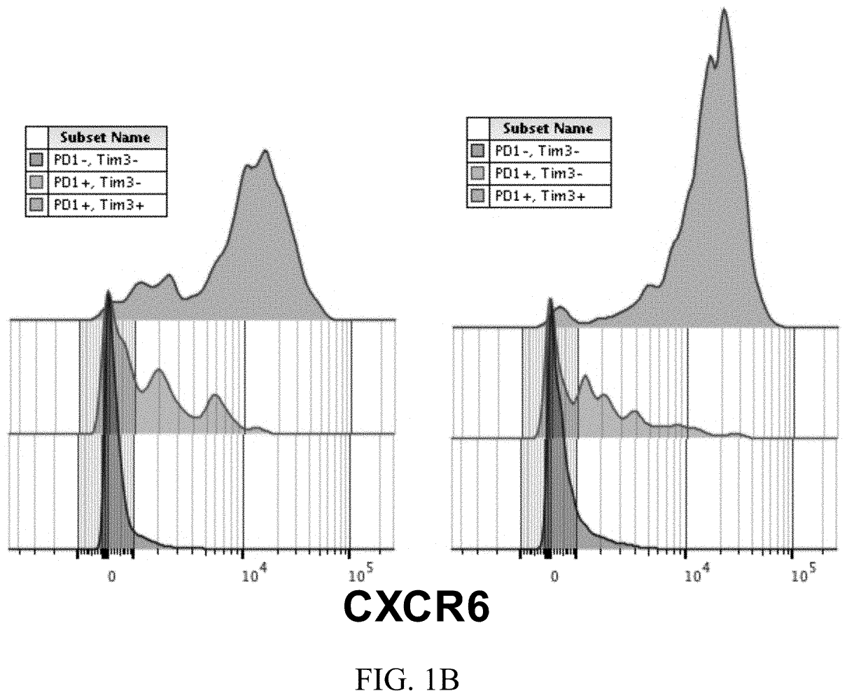

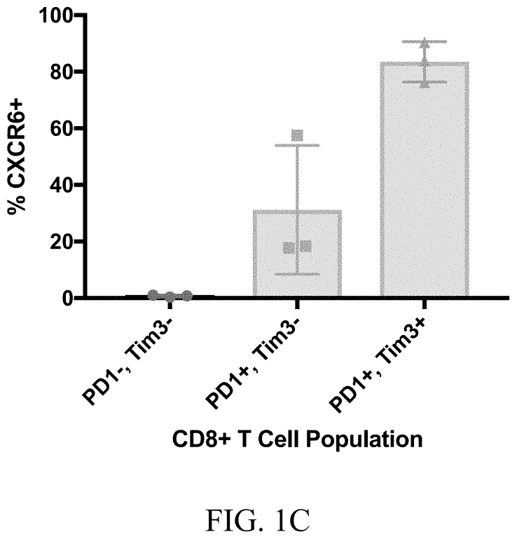

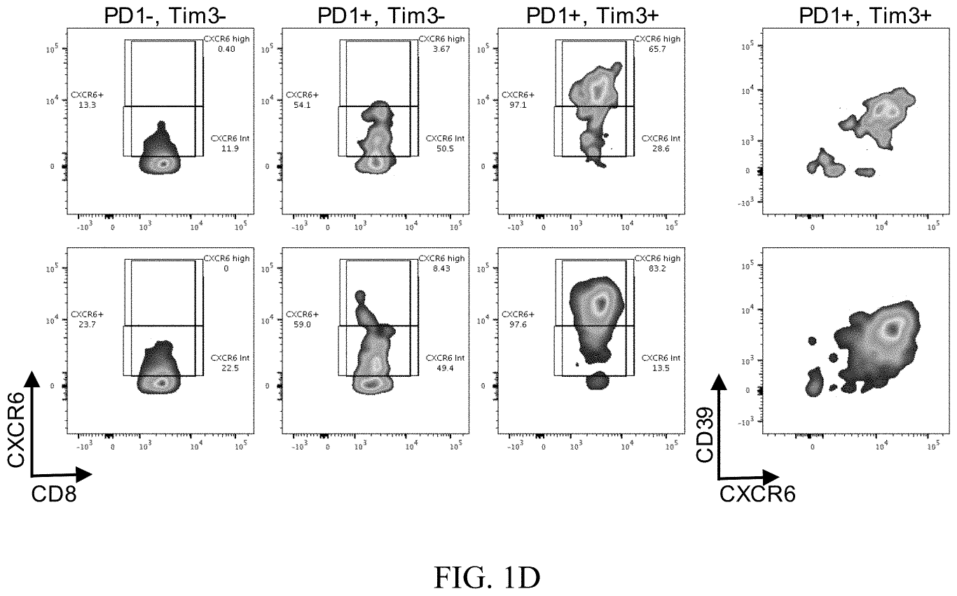

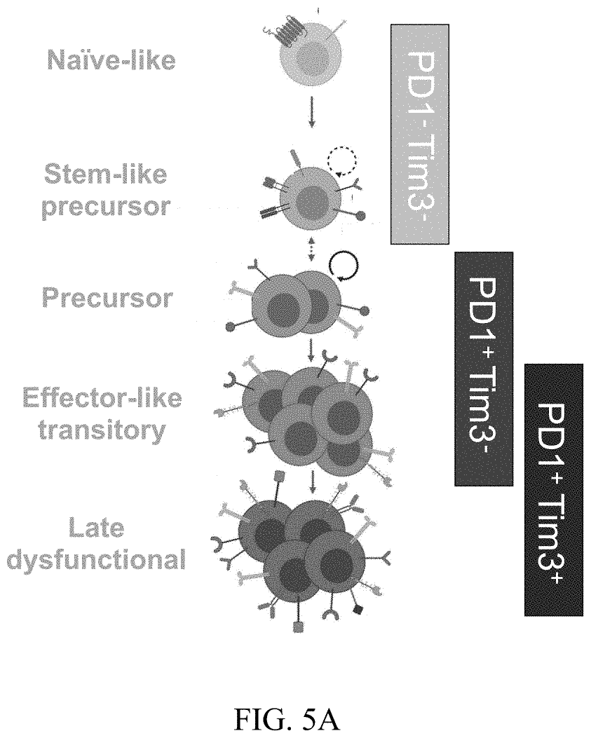

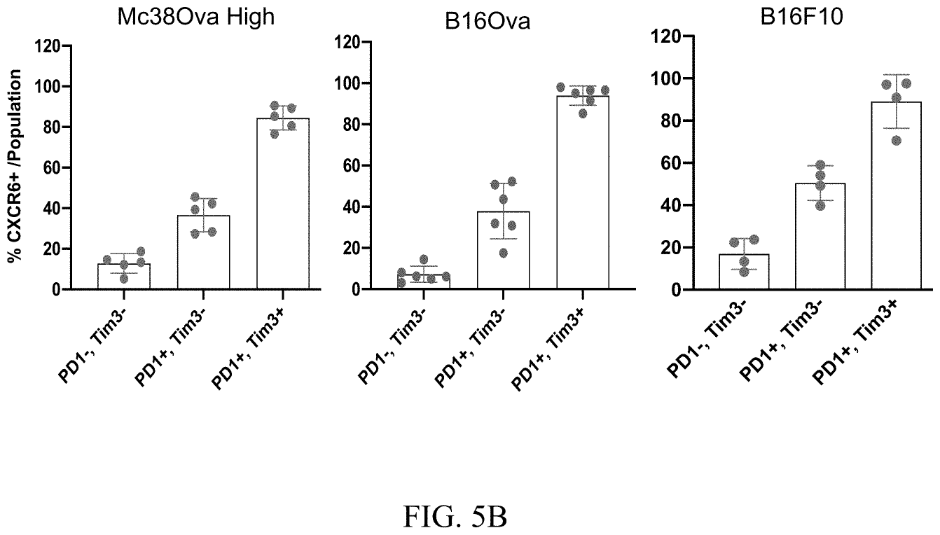

In some embodiments, the CRISPR-Cas system that can be used to modify a polynucleotide of the present invention described herein can be a Class 1 CRISPR-Cas system. Class 1 CRISPR-Cas systems are divided into Types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

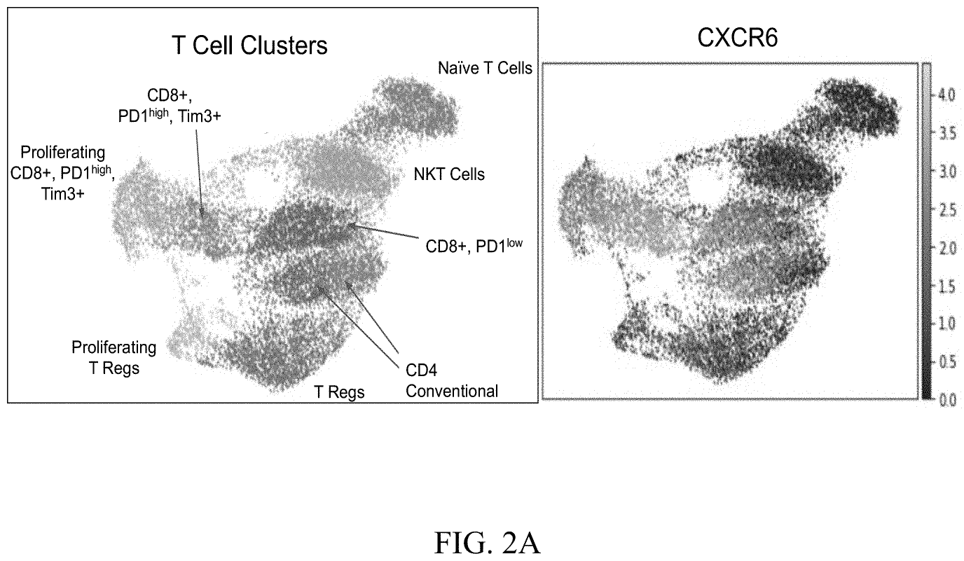

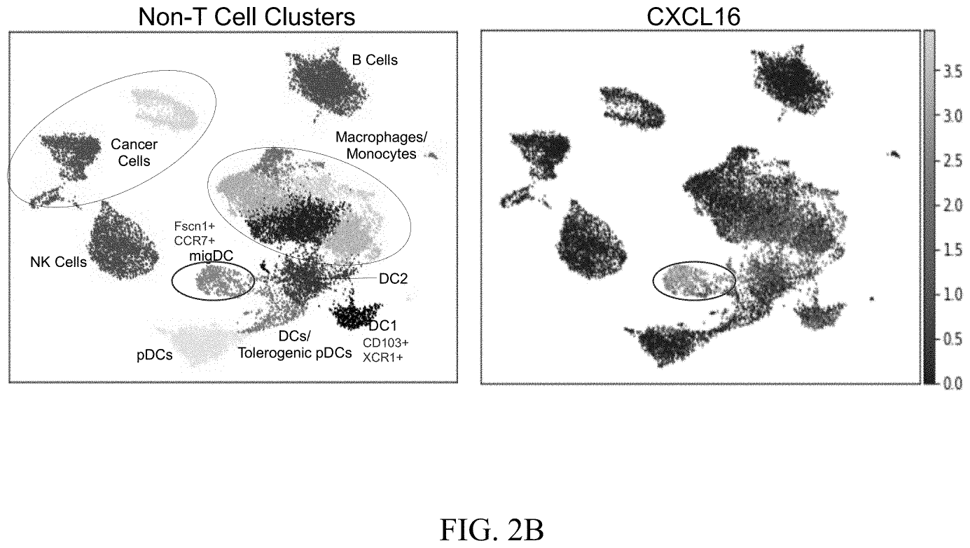

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cash 1). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cash, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1 (V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SETT/9), a translation initiation domain, a transcriptional repression domain (e.g., a KRAB domain, NuE domain, NcoR domain, and a SID domain such as a SID4X domain), a nuclease domain (e.g., Fold), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C•G base pair into a T•A base pair (Komor et al. 2016. Nature. 533:420-424; Nishida et al. 2016. Science. 353; and Li et al. Nat. Biotech. 36:324-327) and ABEs convert an A•T base pair to a G•C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g. A:T to G:C). Richter et al. 2020. Nature Biotechnology.doi.org/10.1038/s41587-020-0453-z.

Other Example Type V base editing systems are described in WO 2018/213708, WO 2018/213726, PCT/US2018/067207, PCT/US2018/067225, and PCT/US2018/067307 which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, WO 2019/005884, WO 2019/005886, WO 2019/071048, PCT/US20018/05179, PCT/US2018/067207, which are incorporated herein by reference. An example FnCas9 system that may be adapted for RNA base editing purposes is described in WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system (See e.g. Anzalone et al. 2019. Nature. 576: 149-157). Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g. sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

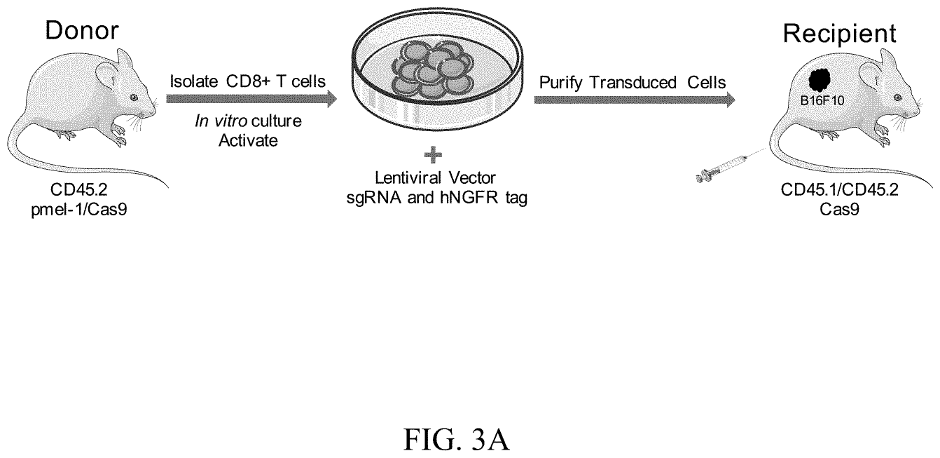

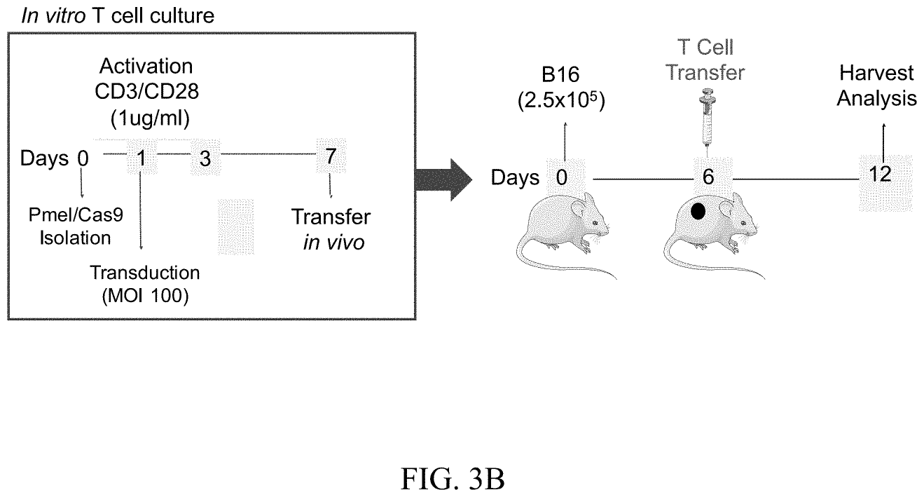

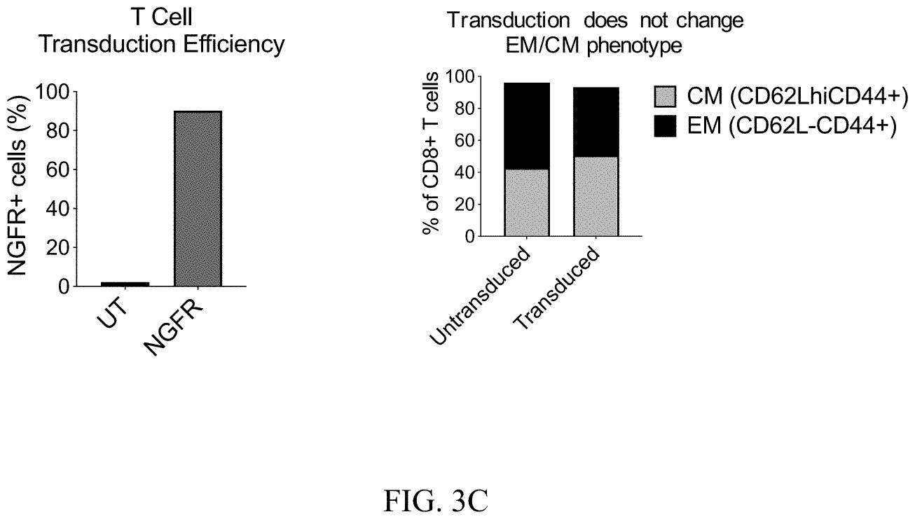

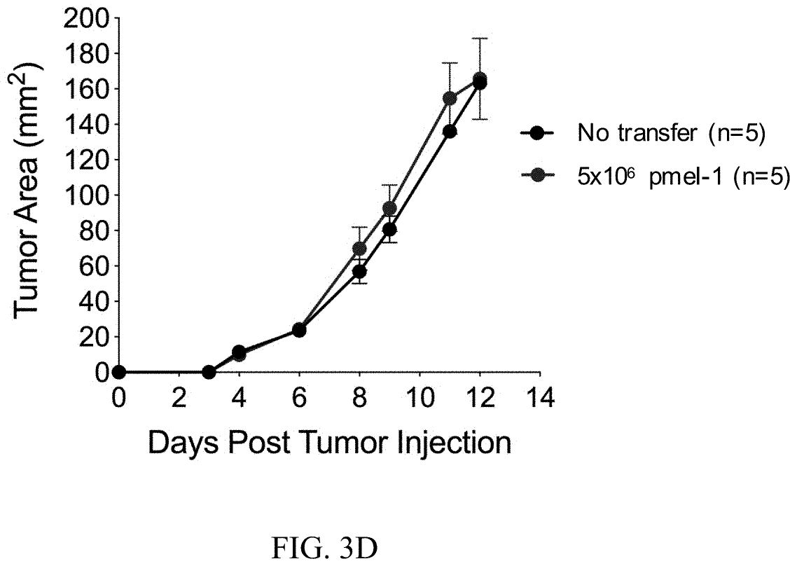

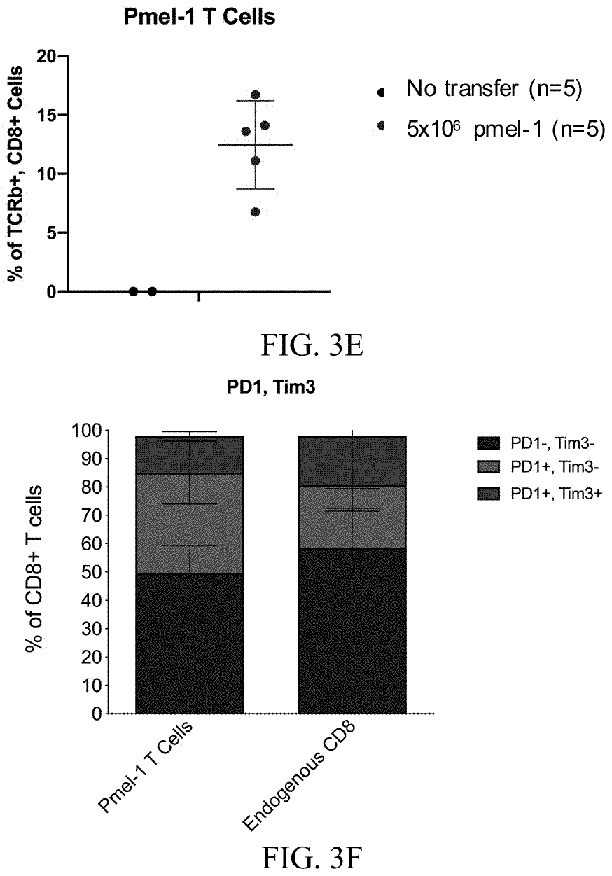

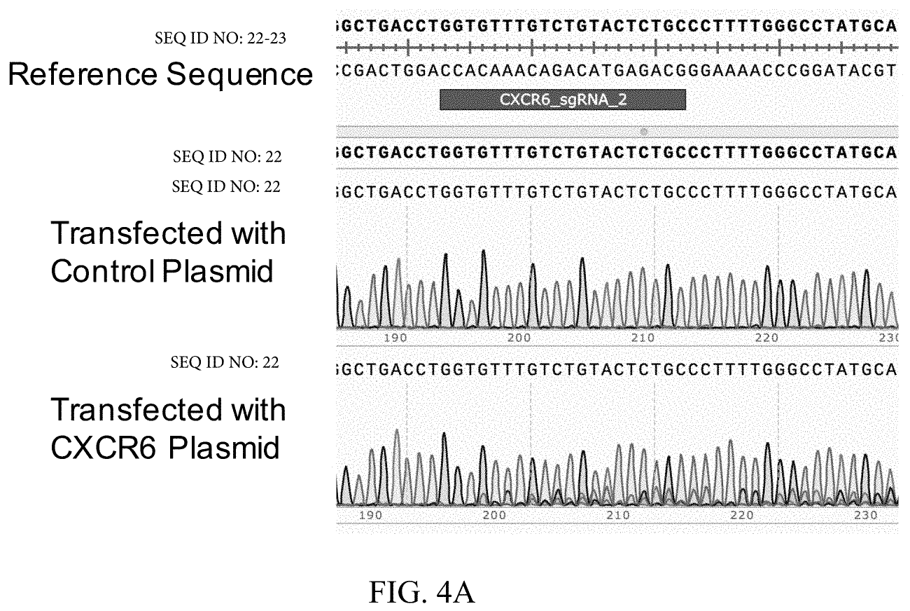

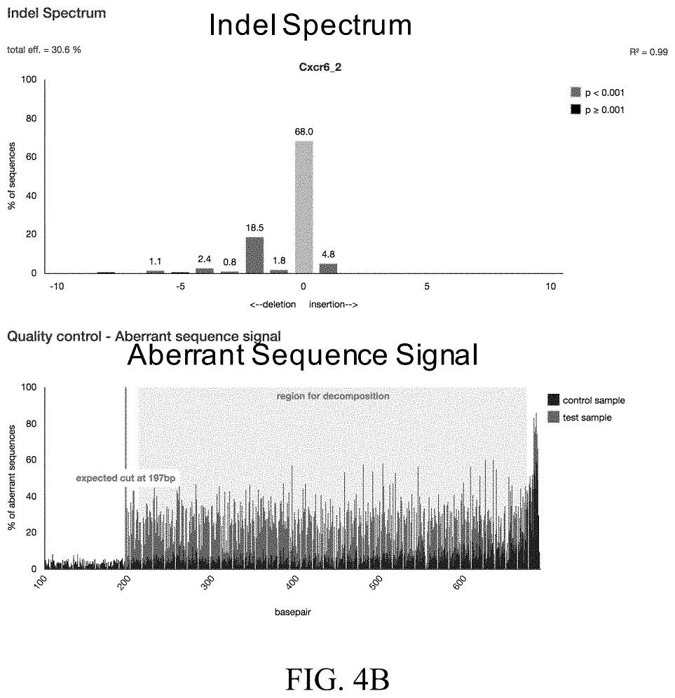

In some embodiments, the prime editing system can be a PE1 system or variant thereof, a PE2 system or variant thereof, or a PE3 (e.g. PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science.aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table A below shows several Cas polypeptides and the PAM sequence they recognize.

TABLE A
Example PAM Sequences

| | |
|---|---|
| Cas Protein | PAM Sequence |
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1)(including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′ end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to FokI cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or ₃₄ or ₃₅, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or ₃₄ or ₃₅)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 3)
M D P I R S R T P S P A R E L L S G P Q P D G V
Q P T A D R G V S P P A G G P L D G L P A R R T
M S R T R L P S P P A P S P A F S A D S F S D L
L R Q F D P S L F N T S L F D S L P P F G A H H
T E A A T G E W D E V Q S G L R A A D A P P P T
M R V A V T A A R P P R A K P A P R R R A A Q P
S D A S P A A Q V D L R T L G Y S Q Q Q Q E K I
K P K V R S T V A Q H H E A L V G H G F T H A H
I V A L S Q H P A A L G T V A V K Y Q D M I A A
L P E A T H E A I V G V G K Q W S G A R A L E A
L L T V A G E L R G P P L Q L D T G Q L L K I A
K R G G V T A V E A V H A W R N A L T G A P L N

An exemplary amino acid sequence of a C-terminal capping region is:

(SEQ ID NO: 4)
R P A L E S I V A Q L S R P D P A L A A L T N D
H L V A L A C L G G R P A L D A V K K G L P H A
P A L I K R T N R R I P E R T S H R V A D H A Q
V V R V L G F F Q C H S H P A Q A F D D A M T Q
F G M S R H G L L Q L F R R V G V T E L E A R S
G T L P P A S Q R W D R I L Q A S G M K R A K P
S P T S T Q T P D Q A S L H A F A D S L E R D L
D A P S P M H E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

### Sequences Related to Nucleus Targeting and Transportation

In some embodiments, one or more components (e.g., the Cas protein and/or deaminase, Zn Finger protein, TALE, or meganuclease) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 5) or PKKKRKVEAS (SEQ ID NO: 6); the NLS from nucleoplasmin (e.g., the nucleoplasmin bipartite NLS with the sequence KRPAATKKAGQAKKKK (SEQ ID NO: 7)); the c-myc NLS having the amino acid sequence PAAKRVKLD (SEQ ID NO: 8) or RQRRNELKRSP (SEQ ID NO: 9); the hRNPA1 M9 NLS having the sequence NQSSNFGPMKGGNFGGRSSGPYGGGGQYFAKPRNQGGY (SEQ ID NO: 10); the sequence RMRIZFKNKGKDTAELRRRRVEVSVELRKAKKDEQILKRRNV (SEQ ID NO: 11) of the IBB domain from importin-alpha; the sequences VSRKRPRP (SEQ ID NO: 12) and PPKKARED (SEQ ID NO: 13) of the myoma T protein; the sequence PQPKKKPL (SEQ ID NO: 14) of human p53; the sequence SALIKKKKKMAP (SEQ ID NO: 15) of mouse c-abl IV; the sequences DRLRR (SEQ ID NO: 16) and PKQKKRK (SEQ ID NO: 17) of the influenza virus NS1; the sequence RKLKKKIKKL (SEQ ID NO: 18) of the Hepatitis virus delta antigen; the sequence REKKKFLKRR (SEQ ID NO: 19) of the mouse Mx1 protein; the sequence KRKGDEVDGVDEVAKKKSKK (SEQ ID NO: 20) of the human poly(ADP-ribose) polymerase; and the sequence RKCLQAGMNLEARKTKK (SEQ ID NO: 21) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g. aptamers) for adapter proteins, which may be linked to or fused to an nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g. due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include sequence that corresponds to both, a first site on the target sequence that is cleaved in a first Cas protein mediated event, and a second site on the target sequence that is cleaved in a second Cas protein mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may be designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

In certain embodiments, a template nucleic acid for correcting a mutation may be designed for use with a homology-independent targeted integration system. Suzuki et al. describe in vivo genome editing via CRISPR/Cas9 mediated homology-independent targeted integration (2016, Nature 540:144-149). Schmid-Burgk, et al. describe use of the CRISPR-Cas9 system to introduce a double-strand break (DSB) at a user-defined genomic location and insertion of a universal donor DNA (Nat Commun. 2016 Jul. 28; 7:12338). Gao, et al. describe “Plug-and-Play Protein Modification Using Homology-Independent Universal Genome Engineering” (Neuron. 2019 Aug. 21; 103(4):583-597).

### RNAi

In some embodiments, the genetic modulating agents may be interfering RNAs. In certain embodiments, diseases caused by a dominant mutation in a gene is targeted by silencing the mutated gene using RNAi. In some cases, the nucleotide sequence may comprise coding sequence for one or more interfering RNAs. In certain examples, the nucleotide sequence may be interfering RNA (RNAi). As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

In certain embodiments, a modulating agent may comprise silencing one or more endogenous genes. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA”, used interchangeably herein, are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Administration of Pharmaceutical Compositions

A “pharmaceutical composition” refers to a composition that usually contains an excipient, such as a pharmaceutically acceptable carrier that is conventional in the art and that is suitable for administration to cells or to a subject.

The pharmaceutical composition according to the present invention can, in one alternative, include a prodrug. When a pharmaceutical composition according to the present invention includes a prodrug, prodrugs and active metabolites of a compound may be identified using routine techniques known in the art. (See, e.g., Bertolini et al., J. Med. Chem., 40, 2011-2016 (1997); Shan et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992)).

The term “pharmaceutically acceptable” as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilizers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavorings, aromatizers, thickeners, agents for achieving a depot effect, coatings, antifungal agents, preservatives, stabilizers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical composition can be applied parenterally, rectally, orally or topically. Preferably, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infusion. The skilled person will understand that compositions which are to be administered orally or topically will usually not comprise cells, although it may be envisioned for oral compositions to also comprise cells, for example when gastro-intestinal tract indications are treated. Each of the cells or active components (e.g., immunomodulants) as discussed herein may be administered by the same route or may be administered by a different route. By means of example, and without limitation, cells may be administered parenterally and other active components may be administered orally.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

In certain embodiments, a pharmaceutical cell preparation as taught herein may be administered in a form of liquid composition. In embodiments, the cells or pharmaceutical composition comprising such can be administered systemically, topically, within an organ or at a site of organ dysfunction or lesion.

Preferably, the pharmaceutical compositions may comprise a therapeutically effective amount of the specified immune cells and/or other active components (e.g., immunomodulants). The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

The amount of the agents which will be effective in the treatment of a particular disorder or condition will depend on the nature of the disorder or condition and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. Effective doses may be extrapolated from dose-response curves derived from in vitro or animal model test systems. Ultimately the attending physician will decide on the appropriate duration of therapy using compositions of the present invention. Dosage will also vary according to the age, weight and response of the individual patient.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

### Diagnostic and Theranostic Applications

The invention provides biomarkers for the identification, diagnosis, prognosis and manipulation of disease phenotypes (e.g., immune state), for use in a variety of diagnostic and/or therapeutic indications. In certain embodiments, biomarkers can be used to detect dysfunctional T cells comprising detecting a dysfunctional gene signature in T cells obtained from a subject in need thereof, wherein the dysfunctional gene signature comprises expression of: one or more genes selected from the group consisting of CXCR6, NDFIP2, CD82, LSP1, FKBP1A, PKM, ACP5, PHLDA1, AKAP5, NAB1, SIRPG, DUSP4, RGS1, GAPDH, RBPJ, TNFRSF9, MIR155HG, CD27, CD2, TNFSF4, CXCL13, SAMSN1, EPSTI1, SARDH, CD74, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1. In certain embodiments, biomarkers increase after treatment (e.g., CPB therapy) and the effectiveness of the treatment can be determined by detecting the biomarkers.

### Pan-Cancer T Cell Exhaustion Regulators

The present invention provides genes upregulated in dysfunctional T cells as compared to non-dysfunctional T cells. These genes can be used for detecting and isolating exhausted T cells. Detection of exhausted T cells can provides for and indication of an immune state. The immune state can be used to determine a diagnosis or for determining the effectiveness of a treatment. As used herein, the terms “exhaustion” and “dysfunction” are used interchangeably. In certain embodiments, a population of CD8+ T cells are modified to be resistant to exhaustion. In certain embodiments, the CD8+ T cells are modified to comprise decreased expression of one or more of exhaustion associated genes. By association is meant that the expression of the genes correlate with an exhaustion phenotype. Correlate may refer to genes that are upregulated or downregulated together. An exhaustion phenotype may include the release or absence of specific cytokines by immune cells. The exhaustion phenotype may be an exhaustion gene signature, described further herein. The T cells may have decreased expression of one or more genes as compared to unmodified T cells. The T cells may be modified to have the expression of one or more exhaustion genes completed abolished. The T cells may be modified to express an agent that decreases or abolishes expression of the one or more genes. The T cells may have one exhaustion gene modified. The T cells may have a combination of exhaustion genes modified, such as 2, 3, 4, 5, 6, 7, 8, 9 or more than 10 exhaustion genes modified. In certain embodiments, exhaustion associated genes may be targeted in vivo to reduce a dysfunction phenotype. In certain embodiments, the expression activity or function of one or more genes is modulated. In certain embodiments, 1, 2, 3, 4, 5, 6, 7, 8, 9 or more than 10 exhaustion genes are targeted. In certain embodiments, exhaustion associated genes are detected to measure an immune response. In certain embodiments, 1, 2, 3, 4, 5, 6, 7, 8, 9 or more than 10 exhaustion genes are detected.

During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8+ or CD4+ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation (e.g., in response to a cytokine, such as IFN-gamma) or cell division, entrance into the cell cycle, cytokine production, cytotoxicity, migration and trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is dysfunctional is a CD8+ T cell that expresses the CD8+ cell surface marker. Such CD8+ cells normally proliferate and produce cell killing enzymes, e.g., they can release the cytotoxins perforin, granzymes, and granulysin. However, exhausted/dysfunctional T cells do not respond adequately to TCR stimulation, and display poor effector function, sustained expression of inhibitory receptors and a transcriptional state distinct from that of functional effector or memory T cells. Dysfunction/exhaustion of T cells thus prevents optimal control of infection and tumors. Exhausted/dysfunctional immune cells, such as T cells, such as CD8+ T cells, may produce reduced amounts of IFN-gamma, TNF-alpha and/or one or more immunostimulatory cytokines, such as IL-2, compared to functional immune cells. Exhausted/dysfunctional immune cells, such as T cells, such as CD8+ T cells, may further produce (increased amounts of) one or more immunosuppressive transcription factors or cytokines, such as IL-10 and/or Foxp3, compared to functional immune cells, thereby contributing to local immunosuppression. Dysfunctional CD8+ T cells can be both protective and detrimental against disease control. As used herein, a “dysfunctional immune state” refers to an overall suppressive immune state in a subject or microenvironment of the subject (e.g., tumor microenvironment). For example, increased IL-10 production leads to suppression of other immune cells in a population of immune cells.

CD8+ T cell function is associated with their cytokine profiles. It has been reported that effector CD8+ T cells with the ability to simultaneously produce multiple cytokines (polyfunctional CD8+ T cells) are associated with protective immunity in patients with controlled chronic viral infections as well as cancer patients responsive to immune therapy (Spranger et al., 2014, J. Immunother. Cancer, vol. 2, 3). In the presence of persistent antigen CD8+ T cells were found to have lost cytolytic activity completely over time (Moskophidis et al., 1993, Nature, vol. 362, 758-761). It was subsequently found that dysfunctional T cells can differentially produce IL-2, TNFa and IFNg in a hierarchical order (Wherry et al., 2003, J. Virol., vol. 77, 4911-4927). Decoupled dysfunctional and activated CD8+ cell states have also been described (see, e.g., Singer, et al. (2016). A Distinct Gene Module for Dysfunction Uncoupled from Activation in Tumor-Infiltrating T Cells. Cell 166, 1500-1511 e1509; WO/2017/075478; and WO/2018/049025).

Dysfunctional T cells may generate a dysfunctional immune response across all immune cells. T cells resistant to dysfunctional may generate an enhanced immune response across all immune cells. In certain embodiments, the present invention provides for modulating immune states. The immune state can be modulated by modulating T cell dysfunction. In certain embodiments, T cells can affect the overall immune state, such as other immune cells in proximity.

The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4⁺, CD8⁺, effector Th, memory Th, regulatory Th, CD4⁺/CD8⁺ thymocytes, CD4−/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

As used throughout this specification, “immune response” refers to a response by a cell of the immune system, such as a B cell, T cell (CD4⁺ or CD8⁺), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4⁺ response or a CD8+ response. Such responses by these cells can include, for example, cytotoxicity, proliferation, cytokine or chemokine production, trafficking, or phagocytosis, and can be dependent on the nature of the immune cell undergoing the response.

T cell response refers more specifically to an immune response in which T cells directly or indirectly mediate or otherwise contribute to an immune response in a subject. T cell-mediated response may be associated with cell mediated effects, cytokine mediated effects, and even effects associated with B cells if the B cells are stimulated, for example, by cytokines secreted by T cells. By means of an example but without limitation, effector functions of MEW class I restricted Cytotoxic T lymphocytes (CTLs) may include cytokine and/or cytolytic capabilities, such as lysis of target cells presenting an antigen peptide recognized by the T cell receptor (naturally-occurring TCR or genetically engineered TCR, e.g., chimeric antigen receptor, CAR), secretion of cytokines, preferably IFN gamma, TNF alpha and/or or more immunostimulatory cytokines, such as IL-2, and/or antigen peptide-induced secretion of cytotoxic effector molecules, such as granzymes, perforins or granulysin. By means of example but without limitation, for MEW class II restricted T helper (Th) cells, effector functions may be antigen peptide-induced secretion of cytokines, preferably, IFN gamma, TNF alpha, IL-4, IL5, IL-10, and/or IL-2. By means of example but without limitation, for T regulatory (Treg) cells, effector functions may be antigen peptide-induced secretion of cytokines, preferably, IL-10, IL-35, and/or TGF-beta. B cell response refers more specifically to an immune response in which B cells directly or indirectly mediate or otherwise contribute to an immune response in a subject. Effector functions of B cells may include in particular production and secretion of antigen-specific antibodies by B cells (e.g., polyclonal B cell response to a plurality of the epitopes of an antigen (antigen-specific antibody response)), antigen presentation, and/or cytokine secretion.

A dynamic regulatory network controls Th17 differentiation (See e.g., Yosef et al., Dynamic regulatory network controlling Th17 cell differentiation, Nature, vol. 496: 461-468 (2013); Wang et al., CD5L/AIM Regulates Lipid Biosynthesis and Restrains Th17 Cell Pathogenicity, Cell Volume 163, Issue 6, p 1413-1427, 3 Dec. 2015; Gaublomme et al., Single-Cell Genomics Unveils Critical Regulators of Th17 Cell Pathogenicity, Cell Volume 163, Issue 6, p 1400-1412, 3 Dec. 2015; and International Patent Publication Nos. WO2016138488A2, WO2015130968, WO/2012/048265, WO/2014/145631 and WO/2014/134351, the contents of which are hereby incorporated by reference in their entirety). As used herein, terms such as “Th17 cell” and/or “Th17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). Depending on the cytokines used for differentiation, in vitro polarized Th17 cells can either cause severe autoimmune responses upon adoptive transfer (‘pathogenic Th17 cells’) or have little or no effect in inducing autoimmune disease (‘nonpathogenic cells’) (Ghoreschi et al., 2010; and Lee et al., 2012 “Induction and molecular signature of pathogenic Th17 cells,” Nature Immunology, vol. 13(10): 991-999). In vitro differentiation of naïve CD4 T cells in the presence of TGF-β1+IL-6 induces an IL-17A and IL-10 producing population of Th17 cells, that are generally nonpathogenic, whereas activation of naïve T cells in the presence of IL-1β+IL-6+IL-23 or TGF-β3+IL-6 induces a T cell population that produces IL-17A and IFN-γ, and are potent inducers of autoimmune disease induction (Ghoreschi et al., 2010, Lee et al., 2012).

As used herein, terms such as “Th1 cell” and/or “Th1 phenotype” and all grammatical variations thereof refer to a differentiated T helper cell that expresses interferon gamma (IFNγ). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

All gene name symbols refer to the gene as commonly known in the art. The examples described herein that refer to the mouse gene names are to be understood to also encompasses human genes, as well as genes in any other organism (e.g., homologous, orthologous genes). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). Any reference to the gene symbol is a reference made to the entire gene or variants of the gene. The signature as described herein may encompass any of the genes described herein. Any reference to a gene is a reference made to the gene and the gene product (e.g., protein).

In certain embodiments, the exhaustion associated genes include one or more of NDFIP2, CD82, LSP1, CXCR6, FKBP1A, PKM, ACP5, PHLDA1, AKAP5, NAB1, SIRPG, DUSP4, RGS1, GAPDH, RBPJ, TNFRSF9, MIR155HG, CD27, CD2, TNFSF4, CXCL13, SAMSN1, EPSTI1, SARDH, CD74, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1.

In certain embodiments, the exhaustion associated genes include checkpoint proteins (described further herein), such as HAVCR2, PDCD1, TIGIT, CTLA4, LAG3, ENTPD1.

In certain embodiments, the exhaustion associated genes include surface proteins, such as CXCR6, TNFRSF9, SIRPG, CD27, CD2, TNFSF4, HLA-DRA, CD8A, HLA-DRB1, HLA-DMA, HLA-DPA1, CD74, TRAF5, BST2, VCAM1, ITGAE, CLEC2D, CD38, ANXA5, CD82, HLA-A, TNFRSF1B, FASLG, PAG1, RAB27A, LY6E, IGFLR1, CD3D and HLA-DRB5. Surface proteins can advantageously be targeted using extracellular therapeutic agents and can be used to identify exhausted cells without breaking of the cells. In certain embodiments, tumors or cells of the tumor microenvironment signal T cells to be dysfunction by binding of ligands or receptors to surface proteins expressed on the T cells.

In certain embodiments, the exhaustion associated genes include secreted proteins, such as ACP5, CXCL13, FAM3C and ISG15. Secreted proteins can advantageously be targeted using extracellular therapeutic agents and can be used to identify a dysfunctional immune state without breaking of the cells. In certain embodiments, tumors or cells of the tumor microenvironment signal induce a dysfunctional immune state by binding of ligands or receptors to secreted proteins expressed from the T cells. In certain embodiments, the secreted proteins are cytokines, in particular chemokines.

In certain embodiments, the exhaustion associated genes include an enzyme, such as PKM, DUSP4 and ACP5. Enzymes can be advantageously targeted or detected based on its substrate or product. The known crystal structure of enzymes can be used to target a binding pocket on the enzyme.

In certain embodiments, the exhaustion associated genes include transcription factors, such as RBPJ, NAB1, TOX, IFI6, ZBED2, IFI16, CCND2, PHLDA1, ETV1

In preferred embodiments, the exhaustion associated genes include one or more of CXCR6, LSP1, CD82, PKM, NDFIP2, FKBP1A and DUSP4.

Exemplary sequences for CXCR6 (BONZO, CD186, STRL33, TYMSTR) include NCBI Reference Sequence: NM_006564.2. In preferred embodiments, the ligand for CXCR6 may include CXCL16 (CXCLG16, SR-PSOX, SRPSOX). CXCR6 has been identified as an entry coreceptor used by HIV-1 and SIV to enter target cells, in conjunction with CD4.

Exemplary sequences for LSP1 (WP34, pp52) include NCBI Reference Sequences: NM_002339.3, NM_001013253.2, NM_001289005.1, NM_001242932.1, NM_001013255.1, and NM_001013254.1. This gene encodes an intracellular F-actin binding protein. The protein is expressed in lymphocytes, neutrophils, macrophages, and endothelium and may regulate neutrophil motility, adhesion to fibrinogen matrix proteins, and transendothelial migration. Alternative splicing results in multiple transcript variants encoding different isoforms.

Exemplary sequences for CD82 (4F9, C33, GR15, IA4, KAI1, R2, SAR2, ST6, TSPAN27) include NCBI Reference Sequences: NM_001024844.2 and NM_002231.4. This metastasis suppressor gene product is a membrane glycoprotein that is a member of the transmembrane 4 superfamily. Expression of this gene has been shown to be downregulated in tumor progression of human cancers and can be activated by p53 through a consensus binding sequence in the promoter. Its expression and that of p53 are strongly correlated, and the loss of expression of these two proteins is associated with poor survival for prostate cancer patients. Two alternatively spliced transcript variants encoding distinct isoforms have been found for this gene.

Exemplary sequences for PKM (CTHBP, HEL-S-30, OIP3, PK3, PKM2, TCB, THBP1, p58) include NCBI Reference Sequences: NM_001206796.3, NM_182470.3, NM_001206797.2, NM_001316318.2, NM_182471.4, NM_001206799.2, NM_002654.6, and NM_001206798.2. This gene encodes a protein involved in glycolysis. The encoded protein is a pyruvate kinase that catalyzes the transfer of a phosphoryl group from phosphoenolpyruvate to ADP, generating ATP and pyruvate. This protein has been shown to interact with thyroid hormone and may mediate cellular metabolic effects induced by thyroid hormones. This protein has been found to bind Opa protein, a bacterial outer membrane protein involved in gonococcal adherence to and invasion of human cells, suggesting a role of this protein in bacterial pathogenesis. Several alternatively spliced transcript variants encoding a few distinct isoforms have been reported.

Exemplary sequences for NDFIP2 (N4WBP5A) include NCBI Reference Sequences: NM_019080.2 and NM_001161407.1. NDFIP2 has been shown to interact with NEDD4.

Exemplary sequences for FKBP1A (FKBP-12, FKBP-1A, FKBP1, FKBP12, PKC12, PKCI2, PPIASE) include NCBI Reference Sequences: NM_001199786.1, NM_054014.3, and NM_000801.5. The protein encoded by this gene is a member of the immunophilin protein family, which play a role in immunoregulation and basic cellular processes involving protein folding and trafficking. The protein is a cis-trans prolyl isomerase that binds the immunosuppressants FK506 and rapamycin. It interacts with several intracellular signal transduction proteins including type I TGF-beta receptor. It also interacts with multiple intracellular calcium release channels, and coordinates multi-protein complex formation of the tetrameric skeletal muscle ryanodine receptor. In mouse, deletion of this homologous gene causes congenital heart disorder known as noncompaction of left ventricular myocardium. Multiple alternatively spliced variants, encoding the same protein, have been identified. The human genome contains five pseudogenes related to this gene, at least one of which is transcribed.

Exemplary sequences for DUSP4 (HVH2, MKP-2, MKP2, TYP) include NCBI Reference Sequences: NM_057158.3 and NM_001394.7. The protein encoded by this gene is a member of the dual specificity protein phosphatase subfamily. These phosphatases inactivate their target kinases by dephosphorylating both the phosphoserine/threonine and phosphotyrosine residues. They negatively regulate members of the mitogen-activated protein (MAP) kinase superfamily (MAPK/ERK, SAPK/JNK, p38), which are associated with cellular proliferation and differentiation. Different members of the family of dual specificity phosphatases show distinct substrate specificities for various MAP kinases, different tissue distribution and subcellular localization, and different modes of inducibility of their expression by extracellular stimuli. This gene product inactivates ERK1, ERK2 and JNK, is expressed in a variety of tissues, and is localized in the nucleus. Two alternatively spliced transcript variants, encoding distinct isoforms, have been observed for this gene. In addition, multiple polyadenylation sites have been reported.

### Gene Signatures

In certain embodiments, a gene signature for use according to any embodiment herein includes one or more of any of the exhaustion associated genes. In certain embodiments, cell types are identified by gene signatures. As used herein a “signature” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. In certain embodiments, a signature includes one or more of CXCR6, LSP1, CD82, PKM, NDFIP2, FKBP1A, and DUSP4. In certain embodiments, a gene signature includes surface proteins. In certain embodiments, a signature includes one or more of CXCR6, LSP1, CD82, PKM, NDFIP2, FKBP1A, and DUSP4; and one or more checkpoint proteins selected from HAVCR2, PDCD1, TIGIT, CTLA4, LAG3 and ENTPD1. In certain embodiments, a gene signature includes one or more surface proteins, one or more checkpoint proteins, one or more transcription factors, and/or one or more secreted proteins.

As used herein, the terms “signature”, “expression profile”, or “expression program” may be used interchangeably. As used herein the term “biological program” or “cell program” may be a type of “signature”, “expression program” or “transcriptional program” and refers to a set of genes that share a role in a biological function (e.g., an activation program, cell differentiation program, proliferation program). Biological programs can include a pattern of gene expression that result in a corresponding physiological event or phenotypic trait. Biological programs can include up to several hundred genes that are expressed in a spatially and temporally controlled fashion. Expression of individual genes can be shared between biological programs. Expression of individual genes can be shared among different single cell types; however, expression of a biological program may be cell type specific or temporally specific (e.g., the biological program is expressed in a cell type at a specific time). Biological programs may be expressed across different cell types. In certain embodiments, a biological program includes genes that co-vary. Expression of a biological program may be regulated by a master switch, such as a nuclear receptor or transcription factor. As used herein, the term “topic” refers to a biological program. The biological program (e.g., topics) can be modeled as a distribution over expressed genes. One method to identify cell programs is non-negative matrix factorization (NMF) (see, e.g., Lee D D and Seung H S, Learning the parts of objects by non-negative matrix factorization, Nature. 1999 Oct. 21; 401(6755):788-91). Other approaches are topic models (Bielecki, Riesenfeld, Kowalczyk, et al., 2018 Skin inflammation driven by differentiation of quiescent tissue-resident ILCs into a spectrum of pathogenic effectors. bioRxiv 461228) and word embeddings. Identifying cell programs can recover cell states and bridge differences between cells. Single cell types may span a range of continuous cell states (see, e.g., Shekhar et al., Comprehensive Classification of Retinal Bipolar Neurons by Single-Cell Transcriptomics Cell. 2016 Aug. 25; 166(5):1308-1323.e30; and Bielecki, et al., 2018 bioRxiv 461228).

It is to be understood that also when referring to proteins (e.g. differentially expressed proteins), such may fall within the definition of “gene” signature. Levels of expression or activity or prevalence may be compared between different cells in order to characterize or identify, for instance, signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein may thus refer to any set of up- and down-regulated genes that are representative of a cell type or subtype. A gene signature as used herein, may also refer to any set of up- and down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. tumor samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory, the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cancer cells that are linked to particular pathological condition (e.g. cancer grade), or linked to a particular outcome or progression of the disease (e.g. metastasis), or linked to a particular response to treatment of the disease.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular cell or cell (sub)population if it is upregulated or only present, detected or detectable in that particular tumor cell or tumor cell (sub)population, or alternatively is downregulated or only absent, or undetectable in that particular tumor cell or tumor cell (sub)population. In this context, a signature consists of one or more differentially expressed genes/proteins or differential epigenetic elements when comparing different cells or cell (sub)populations, including comparing different cells or cell (sub)populations, as well as comparing tumor cells or tumor cell (sub)populations with non-tumor cells or non-tumor cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of tumor cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least to, at least three, at least four, at least five, at least six, or all genes/proteins and/or epigenetic elements of the signature.

Signatures may be functionally validated as being uniquely associated with a particular immune responder phenotype. Induction or suppression of a particular signature may consequentially be associated with or causally drive a particular immune responder phenotype.

Various aspects and embodiments of the invention may involve analyzing gene signatures, protein signature, and/or other genetic or epigenetic signature based on single cell analyses (e.g. single cell RNA sequencing) or alternatively based on cell population analyses, as is defined herein elsewhere.

In further aspects, the invention relates to gene signatures, protein signature, and/or other genetic or epigenetic signature of particular tumor cell subpopulations, as defined herein elsewhere. The invention hereto also further relates to particular tumor cell subpopulations, which may be identified based on the methods according to the invention as discussed herein, as well as methods to obtain such cell (sub)populations and screening methods to identify agents capable of inducing or suppressing particular tumor cell (sub)populations.

The invention further relates to various uses of the gene signatures, protein signature, and/or other genetic or epigenetic signature as defined herein, as well as various uses of the tumor cells or tumor cell (sub)populations as defined herein. Particular advantageous uses include methods for identifying agents capable of inducing or suppressing particular tumor cell (sub)populations based on the gene signatures, protein signature, and/or other genetic or epigenetic signature as defined herein. The invention further relates to agents capable of inducing or suppressing particular tumor cell (sub)populations based on the gene signatures, protein signature, and/or other genetic or epigenetic signature as defined herein, as well as their use for modulating, such as inducing or repressing, a particular gene signature, protein signature, and/or other genetic or epigenetic signature. In one embodiment, genes in one population of cells may be activated or suppressed in order to affect the cells of another population. In related aspects, modulating, such as inducing or repressing, a particular a particular gene signature, protein signature, and/or other genetic or epigenetic signature may modify overall tumor composition, such as tumor cell composition, such as tumor cell subpopulation composition or distribution, or functionality.

The signature genes of the present invention were discovered by analysis of expression profiles of single-cells within a population of cells, thus allowing the discovery of novel cell subtypes that were previously invisible in a population of cells within a tissue. The presence of subtypes may be determined by subtype specific signature genes. The presence of these specific cell types may be determined by applying the signature genes to bulk sequencing data in a patient tumor. Not being bound by a theory, a tumor is a conglomeration of many cells that make up a tumor microenvironment, whereby the cells communicate and affect each other in specific ways. As such, specific cell types within this microenvironment may express signature genes specific for this microenvironment. Not being bound by a theory, the signature genes of the present invention may be microenvironment specific, such as their expression in a tumor. Not being bound by a theory, signature genes determined in single cells that originated in a tumor are specific to other tumors. Not being bound by a theory, a combination of cell subtypes in a tumor may indicate an outcome. Not being bound by a theory, the signature genes can be used to deconvolute the network of cells present in a tumor based on comparing them to data from bulk analysis of a tumor sample. Not being bound by a theory the presence of specific cells and cell subtypes may be indicative of tumor growth, invasiveness and resistance to treatment. The signature gene may indicate the presence of one particular cell type. In one embodiment, the signature genes may indicate that tumor infiltrating T-cells are present. The presence of cell types within a tumor may indicate that the tumor will be resistant to a treatment. In one embodiment, the signature genes of the present invention are applied to bulk sequencing data from a tumor sample obtained from a subject, such that information relating to disease outcome and personalized treatments is determined. In one embodiment, the novel signature genes are used to detect multiple cell states that occur in a subpopulation of tumor cells that are linked to resistance to targeted therapies and progressive tumor growth.

Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein. In certain embodiments, the biomarkers are the genetic variants. In certain embodiments, the biomarkers are genes in a gene module comprising genetic variants. In certain embodiments, the biomarkers are the entire signatures in the gene modules (e.g., including co-varying genes). In certain embodiments, interacting genetic variants or combinations of interacting genetic variants are used in a polygenic risk score for a phenotype.

In certain embodiments, the invention provides uses of the biomarkers for predicting risk for a certain phenotype. In certain embodiments, the invention provides uses of the biomarkers for selecting a treatment. In certain embodiments, a subject having a disease can be classified based on severity of the disease.

The terms “diagnosis” and “monitoring” are commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying specific patient populations based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Hence, the methods may rely on comparing the quantity of biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favorable or unfavorable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterized by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

### Detection of Biomarkers

In one embodiment, the signature genes, biomarkers, and/or cells expressing biomarkers may be detected or isolated by immunofluorescence, immunohistochemistry (IHC), fluorescence activated cell sorting (FACS), mass spectrometry (MS), mass cytometry (CyTOF), sequencing, WGS (described herein), WES (described herein), RNA-seq, single cell RNA-seq (described herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. Detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25). In certain embodiments, cancer is diagnosed, prognosed, or monitored. For example, a tissue sample may be obtained and analyzed for specific cell markers (IHC) or specific transcripts (e.g., RNA-FISH). In one embodiment, tumor cells are stained for cell subtype specific signature genes. In one embodiment, the cells are fixed. In another embodiment, the cells are formalin fixed and paraffin embedded. Not being bound by a theory, the presence of the tumor subtypes indicate outcome and personalized treatments.

The present invention also may comprise a kit with a detection reagent that binds to one or more biomarkers or can be used to detect one or more biomarkers.

### Sequencing

In certain embodiments, sequencing is used to identify expression of genes or transcriptomes in single cells. In certain embodiments, sequencing comprises high-throughput (formerly “next-generation”) technologies to generate sequencing reads. Methods for constructing sequencing libraries are known in the art (see, e.g., Head et al., Library construction for next-generation sequencing: Overviews and challenges. Biotechniques. 2014; 56(2): 61-77). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., cDNA) may include sequencing adaptors that are compatible with use in, e.g., Illumina's reversible terminator method, long read nanopore sequencing, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Schneider and Dekker (Nat Biotechnol. 2012 Apr. 10; 30(4):326-8); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

As used herein the term “transcriptome” refers to the set of transcript molecules. In some embodiments, transcript refers to RNA molecules, e.g., messenger RNA (mRNA) molecules, small interfering RNA (siRNA) molecules, transfer RNA (tRNA) molecules, ribosomal RNA (rRNA) molecules, and complimentary sequences, e.g., cDNA molecules. In some embodiments, a transcriptome refers to a set of mRNA molecules. In some embodiments, a transcriptome refers to a set of cDNA molecules. In some embodiments, a transcriptome refers to one or more of mRNA molecules, siRNA molecules, tRNA molecules, rRNA molecules, in a sample, for example, a single cell or a population of cells. In some embodiments, a transcriptome refers to cDNA generated from one or more of mRNA molecules, siRNA molecules, tRNA molecules, rRNA molecules, in a sample, for example, a single cell or a population of cells. In some embodiments, a transcriptome refers to 25%, 50%, 55, 60, 65, 70, 75, 80, 85, 90, 91, 92, 93, 94, 95, 96, 97, 98, 99, 99.9, or 100% of transcripts from a single cell or a population of cells. In some embodiments, transcriptome not only refers to the species of transcripts, such as mRNA species, but also the amount of each species in the sample. In some embodiments, a transcriptome includes each mRNA molecule in the sample, such as all the mRNA molecules in a single cell.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the present invention involves single cell RNA sequencing (scRNA-seq). In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number Patent Publication No. PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, dimension reduction is used to cluster single cells based on differentially expressed genes. In certain embodiments, the dimension reduction technique may be, but is not limited to, Uniform Manifold Approximation and Projection (UMAP) or t-SNE (see, e.g., Becht et al., Evaluation of UMAP as an alternative to t-SNE for single-cell data, bioRxiv 298430; doi.org/10.1101/298430; and Becht et al., 2019, Dimensionality reduction for visualizing single-cell data using UMAP, Nature Biotechnology volume 37, pages 38-44).

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affibodies, nanobodies, ankyrins, domain antibodies, alternative antibody scaffolds (e.g. diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif.”. (1992).

In certain embodiments, a subject can be categorized based on signature genes or gene programs expressed by a tissue sample obtained from the subject. In certain embodiments, the tissue sample is analyzed by bulk sequencing. In certain embodiments, subtypes can be determined by determining the percentage of specific cell subtypes expressing the identified interacting genetic variants in the sample that contribute to the phenotype. In certain embodiments, gene expression associated with the cells are determined from bulk sequencing reads by deconvolution of the sample. For example, deconvoluting bulk gene expression data obtained from a tumor containing both malignant and non-malignant cells can include defining the relative frequency of a set of cell types in the tumor from the bulk gene expression data using cell type specific gene expression (e.g., cell types may be T cells, fibroblasts, macrophages, mast cells, B/plasma cells, endothelial cells, myocytes and dendritic cells); and defining a linear relationship between the frequency of the non-malignant cell types and the expression of a set of genes, wherein the set of genes comprises genes highly expressed by malignant cells and at most two non-malignant cell types, wherein the set of genes are derived from gene expression analysis of single cells in the tumor or the same tumor type, and wherein the residual of the linear relationship defines the malignant cell-specific (MCS) expression profile (see, e.g., WO 2018/191553; and Puram et al., Cell. 2017 Dec. 14; 171(7):1611-1624.e24).

### Screening for T Cell Modulating Agents

In certain embodiments, the invention provides for screening of therapeutic agents capable of altering exhaustion regulators. In certain embodiments, agents capable of blocking exhaustion regulators on T cells are screened. In certain embodiments, the method comprises: a) applying a candidate agent to a cell population comprising dysfunctional T cells; b) detecting modulation of one or more phenotypic aspects of the cell population by the candidate agent, thereby identifying the agent. The phenotypic aspects of the cell population that is modulated may be a gene signature or biological program specific to a cell type or cell phenotype or phenotype specific to a population of cells (e.g., an anti-tumor immune phenotype). In certain embodiments, steps can include administering candidate modulating agents to cells, detecting identified cell (sub)populations for changes in signatures, or identifying relative changes in cell (sub) populations which may comprise detecting relative abundance of particular gene signatures. The phenotype may be a change in secretion of cytokines associated with dysfunctional or effector T cells. In certain embodiments, candidate agents are screened in vivo models of cancer (e.g., mouse models). In certain embodiments, anti-tumor activity in a model is detected.

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The methods of phenotypic analysis can be utilized for evaluating environmental stress and/or state, for screening of chemical libraries, and to screen or identify structural, syntenic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on immune phenotypes thereof simultaneously in a relatively short amount of time, for example using a high throughput method.

Aspects of the present disclosure relate to the correlation of an agent with the spatial proximity and/or epigenetic profile of the nucleic acids in a sample of cells. In some embodiments, the disclosed methods can be used to screen chemical libraries for agents that modulate chromatin architecture epigenetic profiles, and/or relationships thereof.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present invention may be used to screen for drugs that reduce the signature or biological program in cells as described herein. The signature or biological program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or biological program of the present invention in silico.

In certain embodiments, a pan cancer exhaustion signature can be identified by applying dimensionality reduction on two or more single cell RNA sequencing cohorts comprising dysfunctional T cells simultaneously. Preferably, dimensionality reduction comprises mixed-NMF. In certain embodiments, the dimension reduction technique may be, but is not limited to, Uniform Manifold Approximation and Projection (UMAP) t-SNE, or PHATE (see, e.g., Becht et al., Evaluation of UMAP as an alternative to t-SNE for single-cell data, bioRxiv 298430; doi.org/10.1101/298430; Becht et al., 2019, Dimensionality reduction for visualizing single-cell data using UMAP, Nature Biotechnology volume 37, pages 38-44; and Moon et al., PHATE: A Dimensionality Reduction Method for Visualizing Trajectory Structures in High-Dimensional Biological Data, bioRxiv 120378; doi: doi.org/10.1101/120378). In certain embodiments, the cohorts are from different cancers. In certain embodiments, cohorts from 2, 3, 4, 5, 6, 7, 8, 9, or 10 or more are used. Genes are then identified that characterize both dysfunctional CD8 T cells and regulatory (CD4) T cells. Signature genes for dysfunction and Tregs can be used and are available in the art. RNA velocity is then used to identify genes that are expressed early and/or late during exhaustion.

### Cancer

In certain embodiments, the invention provides for methods and compositions for treating cancer and for methods of detecting an immune state (e.g., for treating cancer). As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of the particular agent chosen, the dosing regimen to be followed, whether it is administered in combination with other compounds, timing of administration, the tissue to be imaged, and the physical delivery system in which it is carried.

For example, in methods for treating cancer in a subject, an effective amount of a combination of agents is any amount that provides an anti-cancer effect, such as reduces or prevents proliferation of a cancer cell or makes a cancer cell responsive to an immunotherapy.

The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The cancer may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

### Kits

In an aspect, the invention provides kits containing any one or more of the elements discussed herein to allow administration of the therapy or detection of exhaustion biomarkers. In certain embodiments, the kit includes reagents to detect at least one gene according to the gene signature as defined in any embodiment herein. For example, primers for detecting gene expression or antibodies for detecting proteins. Elements may be provided individually or in combinations, and may be provided in any suitable container, such as a vial, a bottle, or a tube. In some embodiments, the kit includes instructions in one or more languages, for example in more than one language. In some embodiments, a kit comprises one or more reagents for use in a process utilizing one or more of the elements described herein. Reagents may be provided in any suitable container. For example, a kit may provide one or more delivery or storage buffers. Reagents may be provided in a form that is usable in a particular process, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the kit comprises one or more of the vectors, proteins and/or one or more of the polynucleotides described herein. The kit may advantageously allow the provision of all elements of the systems of the invention.

Further embodiments are illustrated in the following Examples which are given for illustrative purposes only and are not intended to limit the scope of the invention.

### EXAMPLES

### Example 1—Identification of a Human Pan-Cancer T Cell Exhaustion Signature

Applicants analyzed single cell RNA sequencing data from >100 patients, totaling >50,000 cells. The data represents 5 major tumor types: melanoma, breast, lung, colon, and liver cancer. Applicants used mixed-effects computational modeling to score genes for T cell exhaustion (vs. cytotoxicity), clonal expansion, and association with poor immune checkpoint blockade response (Sade-Feldman, M., et al. (2018). Defining T Cell States Associated with Response to Checkpoint Immunotherapy in Melanoma. Cell, 175(4), 998-1013.e20. doi.org/10.1016/j.cell.2018.10.038) (Table 1).

TABLE 1
Pan Cancer CD8+ T cell Exhaustion Marker Analysis
and Immune Checkpoint Inhibitor (ICI) Response

| | No. of supporting datasets | | | Combined p-value (pancancer) | | |
|---|---|---|---|---|---|---|
| | Exhausted | Exhausted vs. | | Exhausted | Exhausted vs. | |
| Gene | vs. effector | naive/memory | Clonal | vs. effector | naive/memory | Clonal |
| RGS1 | 7 | 6 | 2 | 0 | 0 | 9.58E−10 |
| HAVCR2 | 7 | 7 | 4 | 0 | 0 | 0 |
| PDCD1 | 7 | 7 | 4 | 0 | 0 | 0 |
| GAPDH | 7 | 6 | 3 | 0 | 0 | 0 |
| CXCR6 | 7 | 6 | 2 | 0 | 0 | 0 |
| TIGIT | 7 | 7 | 4 | 0 | 0 | 0 |
| RBPJ | 7 | 4 | 3 | 0 | 0 | 0 |
| DUSP4 | 7 | 5 | 3 | 0 | 0 | 0 |
| TNFRSF9 | 7 | 6 | 4 | 0 | 0 | 0 |
| MIR155HG | 7 | 6 | 4 | 0 | 0 | 0 |
| SIRPG | 7 | 5 | 3 | 0 | 0 | 1.56E−08 |
| CTLA4 | 7 | 6 | 4 | 0 | 0 | 3.26E−14 |
| CD27 | 7 | 6 | 3 | 0 | 0 | 1.03E−05 |
| CD2 | 7 | 5 | 2 | 0 | 0 | 0 |
| TNFSF4 | 6 | 5 | 3 | 0 | 0 | 0 |
| CXCL13 | 6 | 4 | 4 | 0 | 0 | 0 |
| SAMSN1 | 6 | 6 | 2 | 0 | 0 | 1.93E−05 |
| EPSTI1 | 6 | 4 | 1 | 0 | 0 | 8.50E−10 |
| SARDH | 6 | 4 | 3 | 0 | 0 | 2.30E−14 |
| APOBEC3C | 6 | 7 | 2 | 0 | 0 | 0 |
| HLA-DRA | 6 | 5 | 4 | 0 | 0 | 0 |
| LAG3 | 6 | 6 | 3 | 0 | 0 | 0 |
| NAB1 | 6 | 4 | 4 | 0 | 0 | 7.29E−06 |
| CD8A | 6 | 6 | 4 | 0 | 0 | 0 |
| PKM | 6 | 5 | 4 | 0 | 0 | 1.61E−09 |
| ACP5 | 6 | 6 | 3 | 0 | 0 | 2.34E−11 |
| ENTPD1 | 6 | 6 | 4 | 0 | 0 | 0 |
| PHLDA1 | 6 | 6 | 3 | 0 | 0 | 5.48E−10 |
| LSP1 | 6 | 6 | 4 | 0 | 0 | 0 |
| NDFIP2 | 6 | 6 | 3 | 0 | 0 | 5.72E−07 |
| HLA-DRB1 | 6 | 7 | 4 | 0 | 0 | 0 |
| TNS3 | 5 | 4 | 3 | 0 | 0 | 2.02E−13 |
| FUT8 | 5 | 5 | 3 | 0 | 0 | 4.04E−07 |
| HLA-DMA | 5 | 4 | 4 | 0 | 0 | 0 |
| TOX | 5 | 5 | 3 | 0 | 0 | 0 |
| FKBP1A | 5 | 6 | 2 | 0 | 0 | 1.25E−06 |
| GOLIM4 | 5 | 5 | 3 | 0 | 0 | 1.33E−15 |
| IFI6 | 5 | 6 | 2 | 0 | 0 | 6.06E−10 |
| LYST | 5 | 6 | 4 | 0 | 0 | 5.77E−15 |
| HLA-DPA1 | 5 | 6 | 3 | 0 | 0 | 0 |
| FAM3C | 5 | 6 | 2 | 0 | 0 | 0.000119556 |
| ZBED2 | 5 | 4 | 2 | 0 | 0 | 0.001937874 |
| CD74 | 5 | 7 | 3 | 0 | 0 | 0 |
| PAG1 | 5 | 4 | 2 | 0 | 0 | 0.002590605 |
| TRAF5 | 5 | 4 | 2 | 0 | 0 | 2.85E−05 |
| RAB27A | 5 | 5 | 3 | 0 | 0 | 0 |
| BST2 | 5 | 4 | 2 | 0 | 0 | 2.41E−14 |
| CLEC2D | 5 | 4 | 0 | 0 | 0 | 0.274411998 |
| CD38 | 5 | 6 | 2 | 0 | 0 | 5.49E−05 |
| AKAP5 | 5 | 6 | 3 | 0 | 0 | 2.72E−13 |
| LY6E | 5 | 5 | 2 | 0 | 0 | 0 |
| VCAM1 | 5 | 6 | 4 | 0 | 0 | 0 |
| ITGAE | 5 | 4 | 3 | 0 | 0 | 0 |
| ISG15 | 5 | 5 | 2 | 0 | 0 | 1.11E−16 |
| XAF1 | 5 | 4 | 1 | 0 | 0 | 5.56E−05 |
| ANXA5 | 5 | 5 | 2 | 0 | 0 | 0 |
| CD82 | 4 | 4 | 2 | 0 | 0 | 6.73E−10 |
| IFI16 | 4 | 5 | 2 | 0 | 0 | 1.23E−14 |
| RHOA | 4 | 4 | 2 | 1.11E−16 | 0 | 0 |
| HLA-A | 4 | 4 | 3 | 0 | 0 | 0 |
| LINC00158 | 4 | 4 | 4 | 1.11E−16 | 0 | 2.26E−12 |
| CCND2 | 4 | 5 | 2 | 0 | 0 | 3.80E−05 |
| TNFRSF1B | 4 | 6 | 4 | 0 | 0 | 7.64E−13 |
| SHFM1 | 4 | 4 | 1 | 0 | 0 | 1.67E−13 |
| GBP5 | 4 | 5 | 1 | 0 | 0 | 2.23E−10 |
| TNIP3 | 4 | 6 | 2 | 0 | 0 | 0 |
| TYMP | 4 | 6 | 2 | 0 | 0 | 0 |
| PLSCR1 | 4 | 4 | 1 | 0 | 0 | 3.49E−06 |
| MX1 | 4 | 4 | 1 | 0 | 0 | 1.28E−06 |
| GBP2 | 4 | 4 | 1 | 0 | 0 | 0.009687336 |
| UBC | 4 | 4 | 2 | 3.25E−12 | 0 | 0 |
| FASLG | 4 | 7 | 2 | 0 | 0 | 0 |
| SNAP47 | 4 | 5 | 4 | 0 | 0 | 0 |
| GALM | 4 | 5 | 2 | 0 | 0 | 4.11E−15 |
| IGFLR1 | 4 | 4 | 2 | 0 | 0 | 3.38E−07 |
| SH2D2A | 4 | 5 | 1 | 5.77E−15 | 0 | 0.000658323 |
| MYO7A | 4 | 4 | 3 | 0 | 0 | 0 |
| CD3D | 4 | 5 | 3 | 0 | 0 | 1.99E−09 |
| AFAP1L2 | 4 | 6 | 3 | 1.11E−16 | 0 | 4.94E−14 |
| HLA-DRB5 | 4 | 6 | 4 | 0 | 0 | 0 |

| | | | ICI response in melanoma (Sade et al. 2018) | | |
|---|---|---|---|---|---|
| | | | | Higher in | Higher in non- |
| | B16 CD8 (p-value) | | | responders (p- | responders (p- |
| Gene | SP vs. DN | DP vs. DN | HLM zscore | value) | value) |
| RGS1 | 7.92E−01 | 1.42E−01 | −1.25 | 1 | 1.71E−45 |
| HAVCR2 | 2.72E−02 | 4.86E−03 | −2.70 | 1 | 6.32E−53 |
| PDCD1 | 9.18E−02 | 1.48E−03 | −2.85 | 1 | 8.43E−49 |
| GAPDH | 9.86E−03 | 4.63E−04 | −2.81 | 1 | 1.33E−51 |
| CXCR6 | 3.38E−01 | 8.46E−04 | −2.04 | 1 | 2.30E−23 |
| TIGIT | 2.84E−01 | 7.35E−04 | −2.28 | 1 | 9.49E−31 |
| RBPJ | 2.85E−02 | 2.37E−03 | −1.27 | 1 | 7.21E−19 |
| DUSP4 | 9.01E−03 | 1.43E−04 | −1.29 | 1 | 7.75E−31 |
| TNFRSF9 | 3.98E−02 | 1.26E−03 | −1.54 | 1 | 3.09E−29 |
| MIR155HG | NA | NA | −1.99 | 1 | 7.12E−53 |
| SIRPG | NA | NA | −1.10 | 1 | 1.00E−17 |
| CTLA4 | 4.49E−02 | 1.34E−03 | −1.40 | 1 | 2.50E−59 |
| CD27 | 1.45E−01 | 8.58E−02 | −1.14 | 1 | 1.43E−13 |
| CD2 | 2.49E−01 | 9.70E−01 | −0.98 | 1 | 2.16E−06 |
| TNFSF4 | 1.22E−01 | 1.64E−02 | −1.64 | 1 | 1.31E−26 |
| CXCL13 | 3.53E−01 | 2.04E−01 | −2.01 | 1 | 8.98E−64 |
| SAMSN1 | 1.09E−01 | 1.01E−01 | −0.50 | 1 | 1.09E−08 |
| EPSTI1 | 8.29E−01 | 9.78E−01 | −2.90 | 1 | 1.08E−33 |
| SARDH | 3.70E−01 | 6.97E−01 | −1.17 | 1 | 8.08E−07 |
| APOBEC3C | NA | NA | −1.35 | 1 | 4.75E−30 |
| HLA-DRA | NA | NA | −1.32 | 1 | 1.37E−23 |
| LAG3 | 4.29E−01 | 5.27E−02 | −3.36 | 1 | 2.44E−24 |
| NAB1 | 2.70E−01 | 2.93E−01 | −0.89 | 1 | 1.61E−12 |
| CD8A | 5.20E−01 | 1.20E−01 | −1.47 | 1 | 8.37E−17 |
| PKM | 3.13E−03 | 1.95E−04 | −1.78 | 1 | 2.11E−21 |
| ACP5 | 8.64E−01 | 9.73E−01 | −2.10 | 1 | 2.40E−11 |
| ENTPD1 | 8.67E−01 | 1.83E−01 | −1.17 | 1 | 4.87E−60 |
| PHLDA1 | 5.51E−01 | 1.15E−01 | −1.05 | 1 | 2.46E−20 |
| LSP1 | 4.12E−01 | 6.79E−02 | −2.33 | 1 | 3.56E−38 |
| NDFIP2 | 1.32E−01 | 4.26E−04 | NA | NA | NA |
| HLA-DRB1 | NA | NA | −2.43 | 1 | 4.84E−35 |
| TNS3 | 7.00E−01 | 3.58E−02 | NA | NA | NA |
| FUT8 | 1.77E−01 | 8.04E−01 | −0.98 | 1 | 1.16E−07 |
| HLA-DMA | NA | NA | −0.85 | 1 | 8.09E−07 |
| TOX | 7.22E−03 | 1.80E−02 | −1.74 | 1 | 1.39E−26 |
| FKBP1A | 4.88E−02 | 7.66E−03 | −1.83 | 1 | 5.41E−17 |
| GOLIM4 | 1.98E−01 | 6.12E−01 | −2.92 | 1 | 3.30E−65 |
| IFI6 | NA | NA | −2.90 | 1 | 1.37E−64 |
| LYST | 5.00E−01 | 6.94E−01 | −1.57 | 1 | 8.65E−26 |
| HLA-DPA1 | NA | NA | −1.95 | 1 | 2.45E−23 |
| FAM3C | 8.22E−01 | 5.10E−01 | −1.62 | 1 | 1.88E−22 |
| ZBED2 | NA | NA | NA | NA | NA |
| CD74 | 1.45E−01 | 1.92E−03 | −1.22 | 1 | 3.42E−15 |
| PAG1 | 8.23E−01 | 9.29E−01 | NA | NA | NA |
| TRAF5 | 9.66E−01 | 9.94E−01 | −1.39 | 1 | 9.61E−11 |
| RAB27A | 1.82E−01 | 1.97E−01 | −1.62 | 1 | 1.10E−19 |
| BST2 | 9.58E−01 | 9.91E−01 | −3.34 | 1 | 8.08E−28 |
| CLEC2D | 3.17E−01 | 3.79E−01 | NA | NA | NA |
| CD38 | 9.77E−01 | 9.02E−01 | −2.64 | 1 | 1.97E−50 |
| AKAP5 | 9.88E−01 | 9.92E−01 | −0.98 | 1 | 3.88E−06 |
| LY6E | 9.41E−01 | 9.71E−01 | −1.76 | 1 | 7.31E−20 |
| VCAM1 | 5.33E−01 | 1.24E−01 | −1.68 | 1 | 3.44E−49 |
| ITGAE | 1.40E−01 | 4.18E−02 | NA | NA | NA |
| ISG15 | 1.22E−01 | 5.99E−01 | −2.55 | 1 | 1.19E−31 |
| XAF1 | 3.85E−01 | 8.43E−01 | −2.25 | 1 | 5.88E−31 |
| ANXA5 | 2.65E−02 | 1.85E−02 | −2.15 | 1 | 3.37E−30 |
| CD82 | 1.62E−01 | 6.48E−03 | NA | NA | NA |
| IFI16 | NA | NA | −2.70 | 1 | 1.54E−22 |
| RHOA | 1.16E−01 | 7.20E−02 | −1.72 | 1 | 1.06E−11 |
| HLA-A | NA | NA | −1.61 | 1 | 1.85E−11 |
| LINC00158 | NA | NA | −1.36 | 1 | 7.44E−10 |
| CCND2 | 9.23E−01 | 6.92E−01 | NA | NA | NA |
| TNFRSF1B | 2.98E−01 | 1.10E−02 | −1.99 | 1 | 1.56E−25 |
| SHFM1 | 4.10E−02 | 1.22E−02 | −1.91 | 1 | 6.82E−13 |
| GBP5 | 3.34E−01 | 5.55E−01 | −2.42 | 1 | 4.18E−15 |
| TNIP3 | 1.92E−01 | 1.20E−01 | NA | NA | NA |
| TYMP | 9.65E−01 | 8.70E−01 | −2.79 | 1 | 8.38E−15 |
| PLSCR1 | 2.52E−01 | 2.80E−02 | −2.52 | 1 | 3.77E−16 |
| MX1 | 2.84E−01 | 8.88E−02 | −1.21 | 1 | 6.82E−25 |
| GBP2 | 5.12E−01 | 8.13E−01 | −2.11 | 1 | 9.23E−19 |
| UBC | 8.94E−01 | 8.87E−01 | −1.06 | 1 | 2.85E−16 |
| FASLG | NA | NA | −1.20 | 1 | 1.15E−09 |
| SNAP47 | 8.01E−01 | 9.63E−01 | −2.68 | 1 | 8.00E−30 |
| GALM | 1.92E−01 | 8.48E−02 | −2.38 | 1 | 2.61E−28 |
| IGFLR1 | 5.50E−01 | 4.13E−01 | −1.50 | 1 | 5.83E−10 |
| SH2D2A | 3.34E−01 | 6.85E−02 | −3.36 | 1 | 2.42E−36 |
| MYO7A | 4.57E−01 | 5.41E−01 | −1.51 | 1 | 3.13E−26 |
| CD3D | 4.51E−01 | 6.61E−01 | NA | NA | NA |
| AFAP1L2 | 3.97E−01 | 5.79E−01 | −1.59 | 1 | 2.66E−17 |
| HLA-DRB5 | NA | NA | −2.04 | 1 | 3.50E−27 |

Applicants identified 7 candidate pan-cancer T cell exhaustion targets (CXCR6, LSP1, CD82, PKM, NDFIP2, FKBP1A and DUSP4) and selected CXCR6 for further analysis.

### Example 2—CXCR6 (Bonzo) and CXCL16 Expression in Tumors

CXCR6 is a chemokine receptor that binds CXCL16. Along with being secreted, CXCL16 has a transmembrane domain, mediates cell homing, and may facilitate cell-cell interactions in the tumor microenvironment (TME). CXCR6 has been shown to mediate recruitment of CD8+ T cells into inflamed liver, heart, and joints (Sato, T., Thorlacius, H., Johnston, B., Staton, T. L., Xiang, W., Littman, D. R., & Butcher, E. C. (2004). Role for CXCR6 in Recruitment of Activated CD8+Lymphocytes to Inflamed Liver. The Journal of Immunology, 174(1), 277-283. doi.org/10.4049/jimmunol.174.1.277; Nanki, T., Shimaoka, T., Hayashida, K., Taniguchi, K., Yonehara, S., & Miyasaka, N. (2005). Pathogenic role of the CXCL16-CXCR6 pathway in rheumatoid arthritis. Arthritis & Rheumatism, 52(10), 3004-3014. doi.org/10.1002/art.21301; and Yamauchi, R., Tanaka, M., Kume, N., Minami, M., Kawamoto, T., Togi, K., et al. (2004). Upregulation of SR-PSOX/CXCL16 and Recruitment of CD8+ T Cells in Cardiac Valves During Inflammatory Valvular Heart Disease. Arteriosclerosis, Thrombosis, and Vascular Biology, 24(2), 282-287. doi.org/10.1161/01.ATV.0000114565.42679.c6). CXCR6 deficient mice had no defects in Lysteria infection control, or CD4+ and CD8+ T cell responses. CXCR6 is seen to be part of a core transcriptional signature of human tissue-resident memory cells. CXCR6 is related to memory, clonal deletion, and NKT cells.

Applicants hypothesized that a CXCR6:CXCL16 interaction places CD8+ T cells in a niche that determines their anti-tumor functionality. CD8+ T cell populations were sorted from B16F10 tumors in WT mice. Extracted RNA was submitted for bulk RNA sequencing as described (Singer, M., Wang, C., Cong, Le., et al. (2016). A Distinct Gene Module for Dysfunction Uncoupled from Activation in Tumor-Infiltrating T Cells, Cell, 166(6), 1500-1511.e9. doi.org/10.1016/j.cell.2016.08.052) (FIG. 1A). CXCR6 mRNA expression was highest in PD1+, Tim3+ CD8+ T cells known to have a dysfunctional phenotype.

WT mice were injected with 3×10⁵B16F10 melanoma cells and tumors were harvested on D12. TIL fraction was obtained with a percoll density gradient, and cells were analyzed by flow cytometry (FIG. 1B-D). CXCR6 expression progressively increases in CD8+ T cells with the stepwise accumulation of checkpoint molecules. CXCR6 expression is highest in T cells positive for multiple checkpoint molecules.

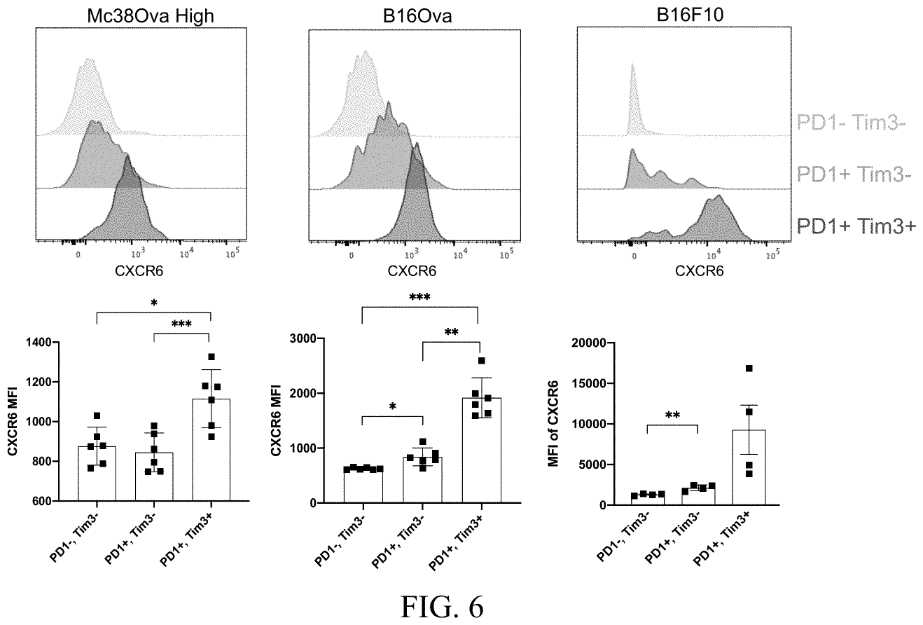

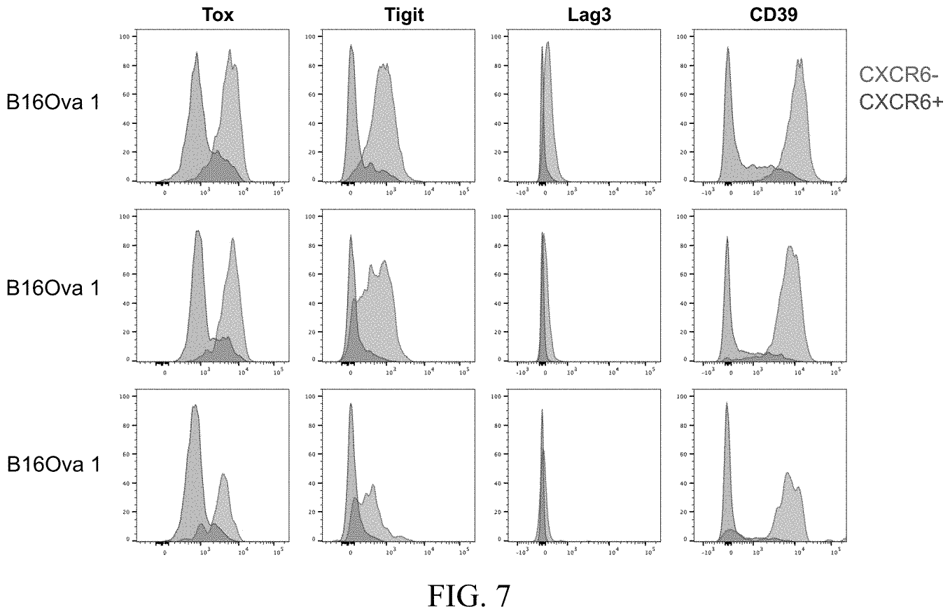

Applicants performed scRNA-sequencing using the 10× platform on CD45+ cells sorted from B16F10 tumors. Applicants used dimension reduction (UMAP) to cluster and annotate T cell and non-T cell clusters (FIGS. 2A and 2B). The B16F10 scRNAseq data shows that Cxcr6 mRNA mirrors the flow cytometric protein data. CXCR6 expression was highest in in PD1+ Tim3+CD8+ TILs. PD1 and Tim3 expression correlates with T cell state with naïve-like and stem-like precursor T cells PD1− TIM3−, late dysfunctional T cells PD1+ Tim3+, and intermediate states PD1+ Tim3− (FIG. 5A). Applicants confirmed that CXCr6+ T cells were expressed highest in the PD1+ Tim3+ cells in three tumor mouse models (FIGS. 5B and 6). Applicants further determined that CXCR6+ cells express many known inhibitory receptors (FIG. 7). Additionally, the B16F10 scRNAseq data shows that Cxcr6 mRNA mirrors the flow cytometric protein data (FIG. 2A).

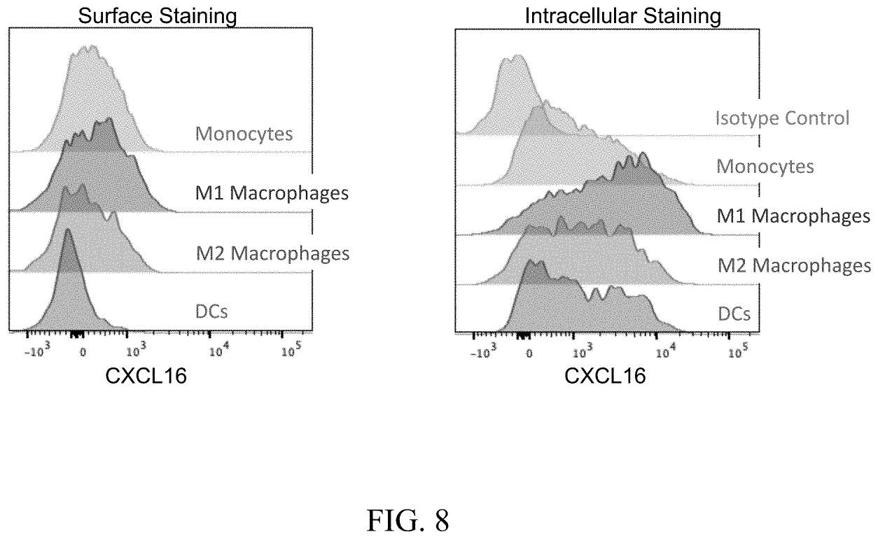

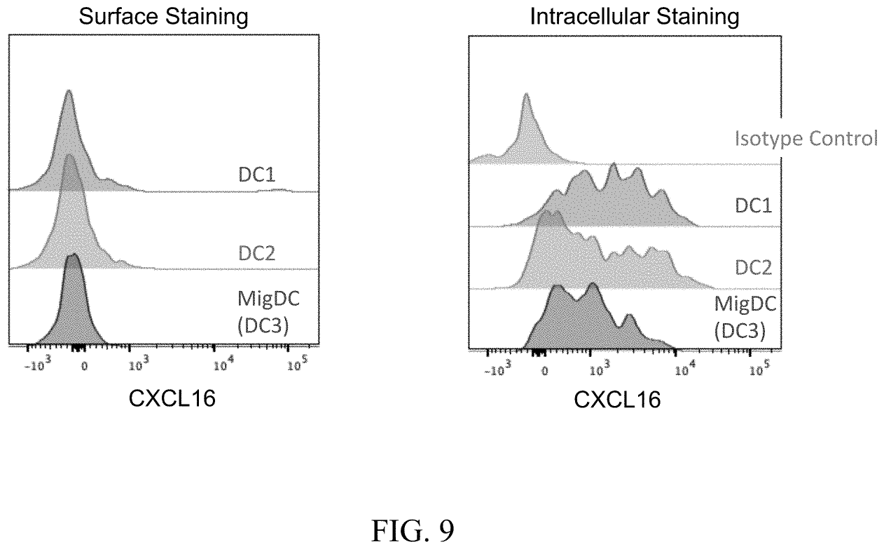

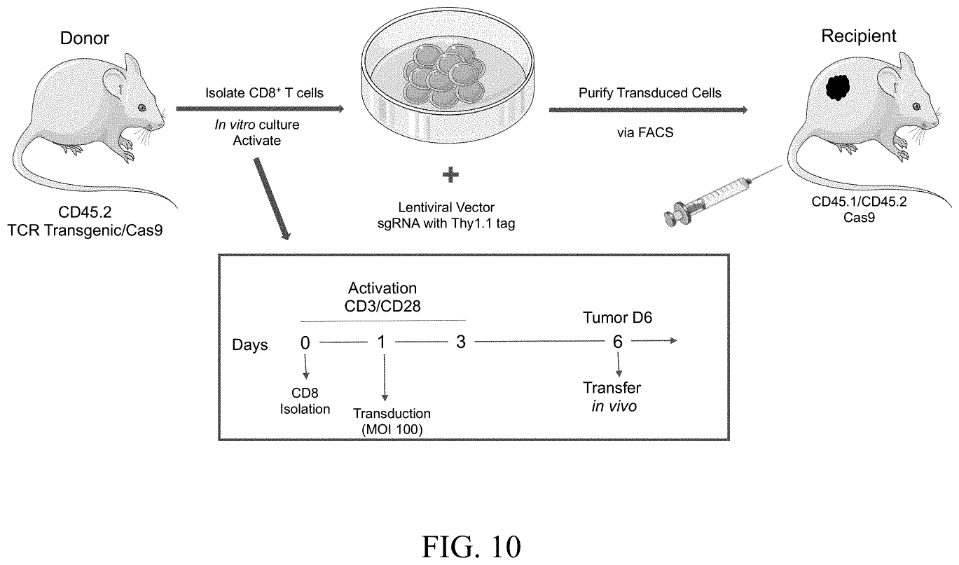

The CXCR6 ligand, CXCL16, is highest in migratory DCs (DC3). Single cell RNA-Sequencing revealed that CXCL16 is highest expressed in a DC population that mediates CD8+ anti-tumor functions and response to immunotherapy (DC3) (FIG. 2B). DC3 dendritic cells have superior cross-presenting abilities, are “activated,” are necessary for anti-tumor immunity, secrete IL-12, and effectively primes T cells responsible for aPD-1 effects (see, e.g., Garris, et al. (2018). Successful Anti-PD-1 Cancer Immunotherapy Requires T Cell-Dendritic Cell Crosstalk Involving the Cytokines IFN-γ and IL-12. Immunity, 49(6), 1148-1161.e7.doi.org/10.1016/j.immuni.2018.09.024; and Zilionis, et al. (2019). Single-Cell Transcriptomics of Human and Mouse Lung Cancers Reveals Conserved Myeloid Populations across Individuals and Species. Immunity, 50(5), 1317-1334.e10. doi.org/10.1016/j.immuni.2019.03.009). CXCR6/CXCL16 T cell/DC interactions may lead to improved T cell responses. CXCL16 was shown to be expressed in other myeloid cells and is mostly intracellular (FIG. 8). Additionally, CXCL16 is expressed in all dendritic cell (DC) subsets (FIG. 9). This highlights a difference between flow cytometric data which measures protein and scRNAseq which measures mRNA transcripts.

### Example 3—T Cell CRISPR/Cas9 KO Transfer with Pmel-1/B16F10 Melanoma Model

Applicants set up a system for testing anti-tumor immunity using CD8+ T cells edited at the candidate exhaustion genes using CRISPR (FIG. 3A-B, 10). Applicants validated the system by showing high transduction efficiency using the NGFR marker, as well as no change in the EM/CM phenotype (FIG. 3C). Applicants show that transferred pmel-1 CD8+ T cells fail to inhibit tumor growth (FIG. 3D). Applicants show that transferred pmel-1 CD8+ T cells are found in the tumor (FIG. 3E). Applicants show similar PD1, Tim3 trajectories in endogenous and transferred pmel-1 CD8+ T cells (FIG. 3F). Applicants can use the system to test whether deleting genes that contribute to T cell exhaustion lead to increased tumor control in mice receiving the CRISPR′ ed CD8+ T cells.

### Example 4—CXCR6 T Cell CRISPR/Cas9 KO Transfer

In order to have the cleanest CRISPR knockout, Applicants chose the sgRNA guide that is most efficient. Using the Broad sgRNA guide design tool, Applicants produced 3 different CXCR6 targeting sgRNA plasmids to test. Applicants transfected a 3T3/inducible-Cas9 murine cell line with the different guides and assessed CRISPR efficiency using the TIDE CRISPR web tool. Applicants identified the guide that elicited the most efficient editing (FIG. 4A-B).

Applicants performed pmel-1 transfers of CXCR6 deleted vs. control into B16F10 to observe tumor growth kinetics. Applicants assessed cytokine production from CD8+ T cells in relation to CXCR6 expression. Applicants used a scRNA-sequencing dataset from a B16F10 time course to evaluate CXCR6 and CXCL16 expression over tumor progression. Applicants examined CXCR6 and CXCL16 spatial expression in the TME using CODEX for multiplexed tissue imaging. Applicants can assess CXCR6 CD8+ T cell expression in tumors undergoing checkpoint blockade therapy. Applicants analyzed CXCR6 expression and correlations with checkpoint molecules in human colorectal carcinoma tissue.

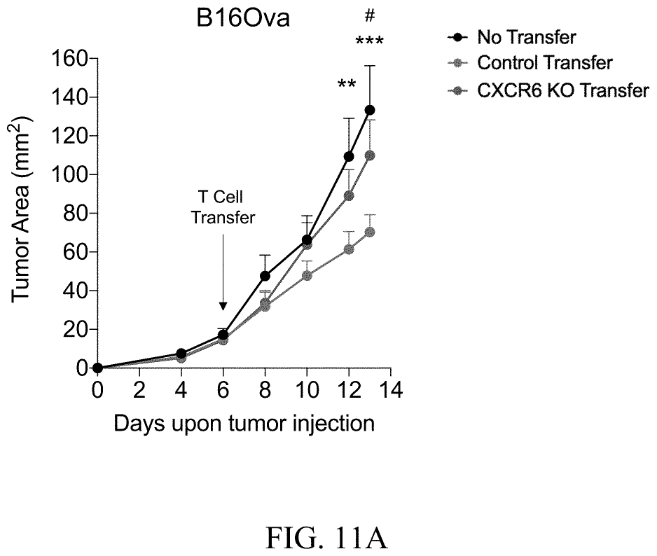

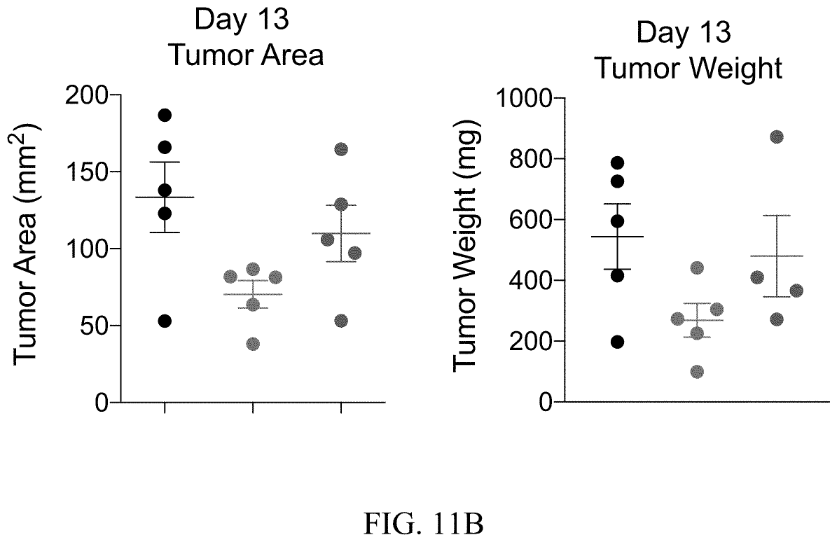

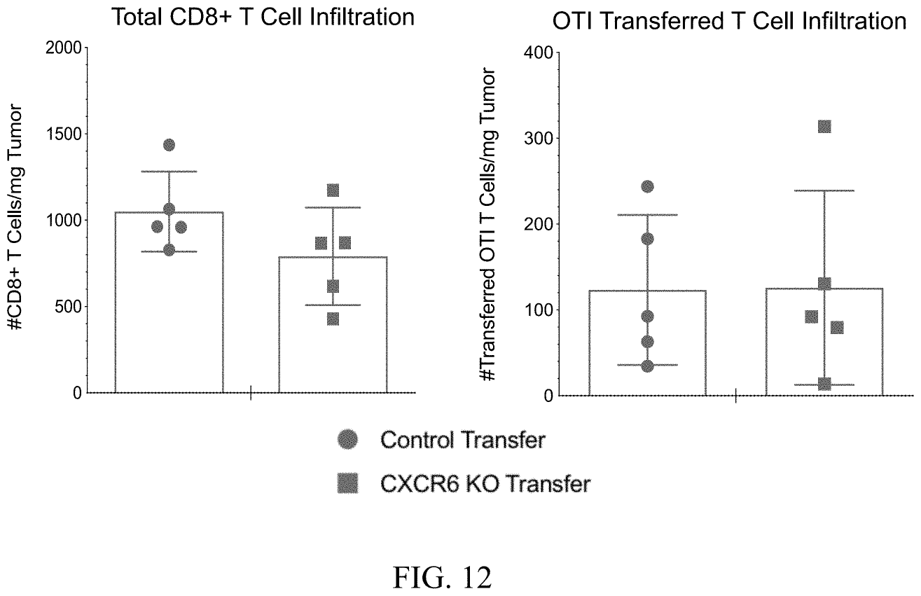

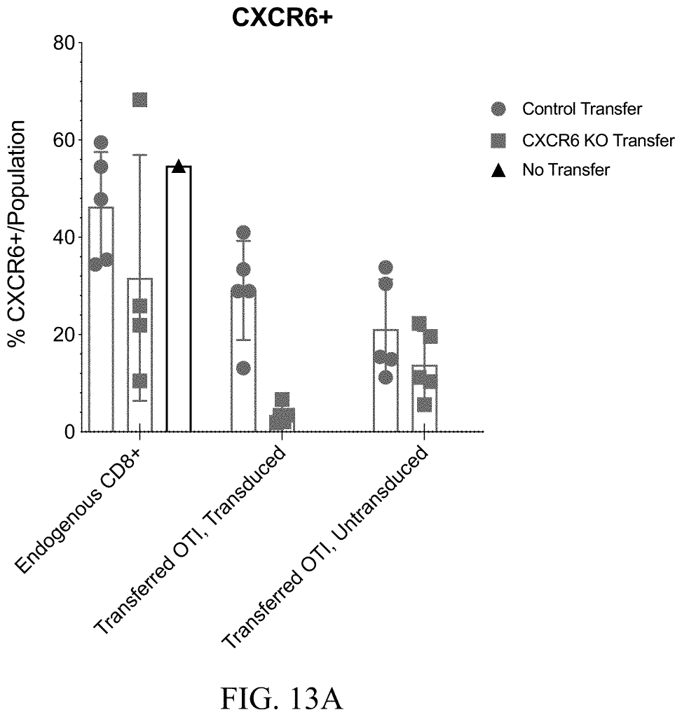

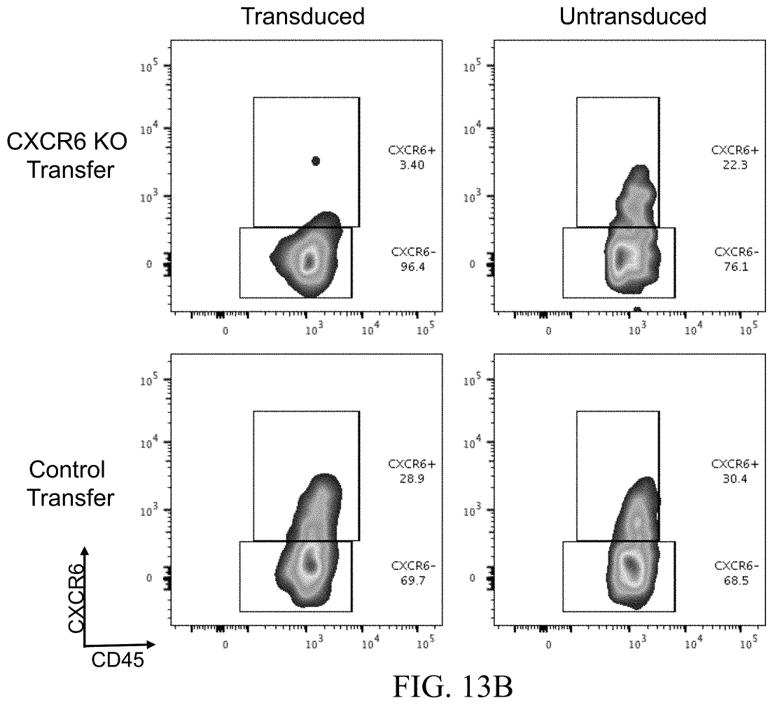

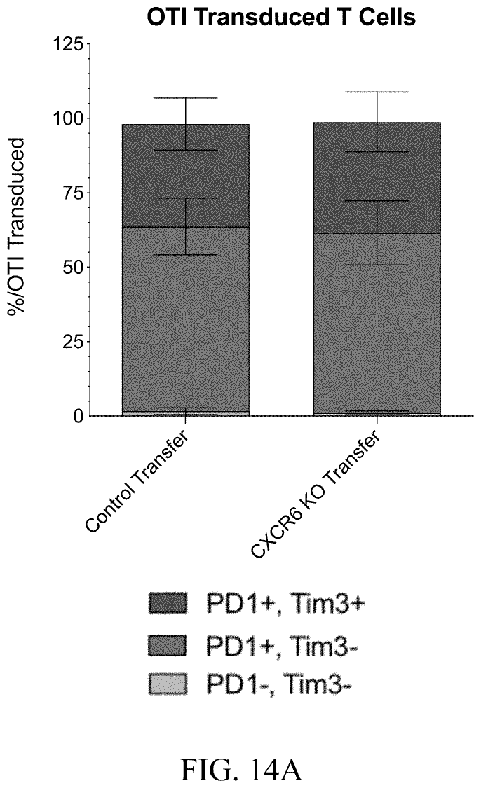

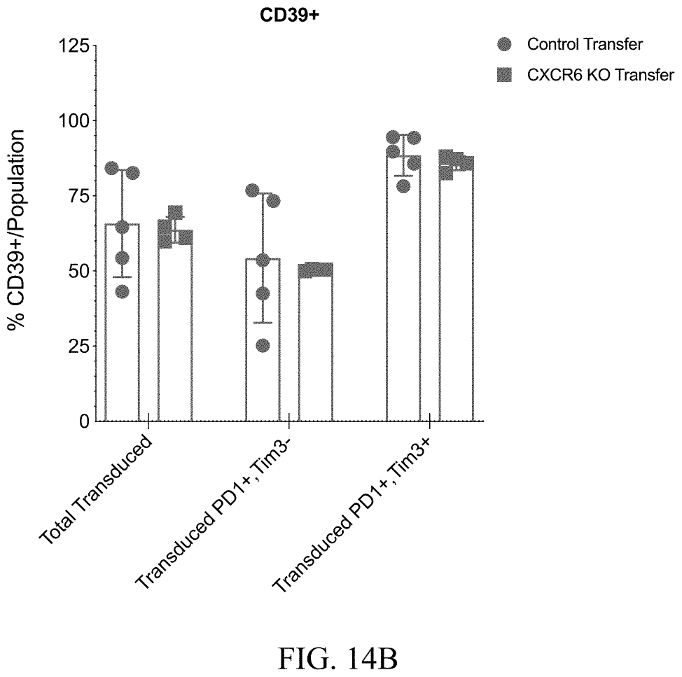

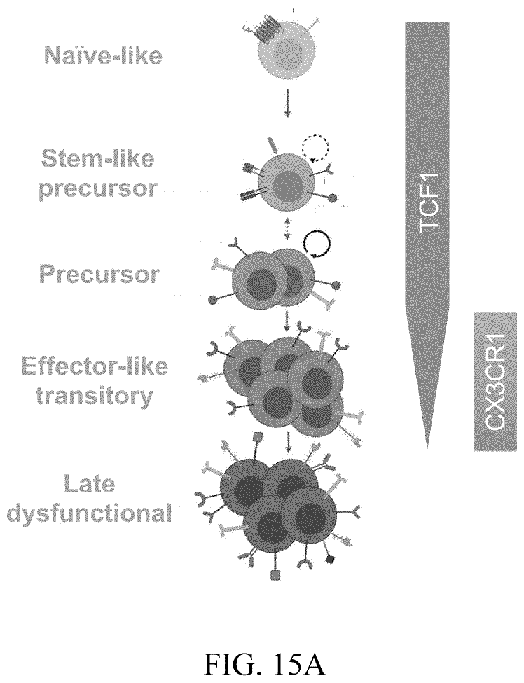

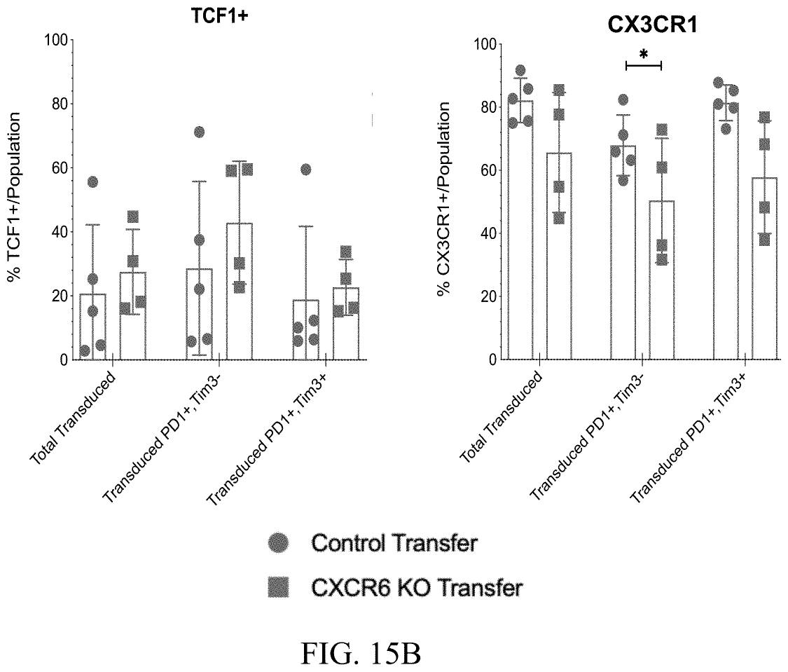

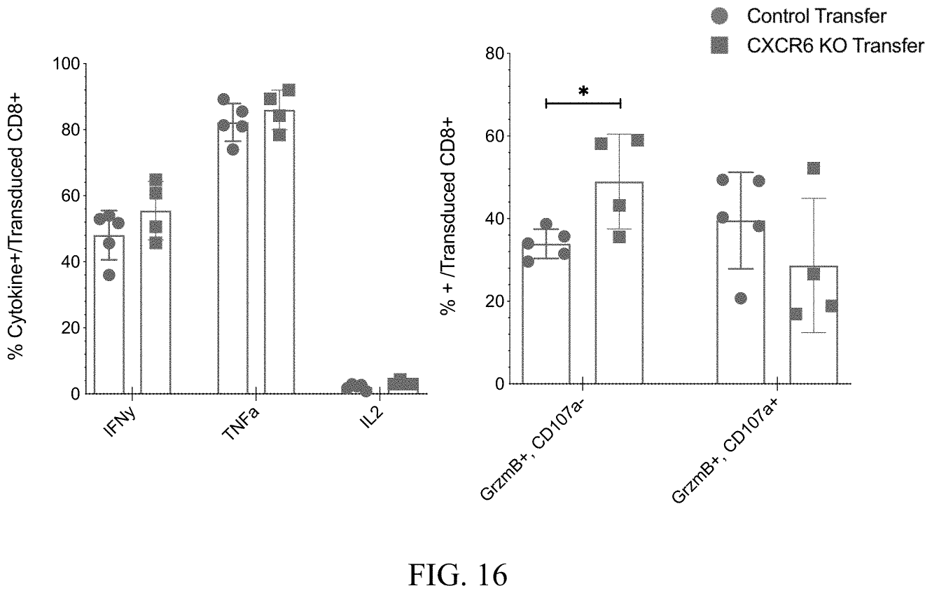

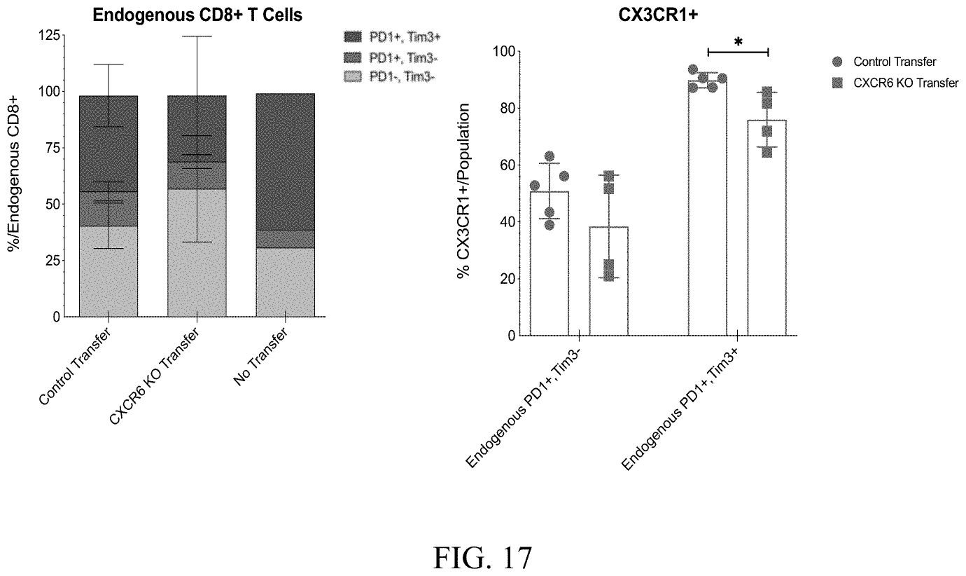

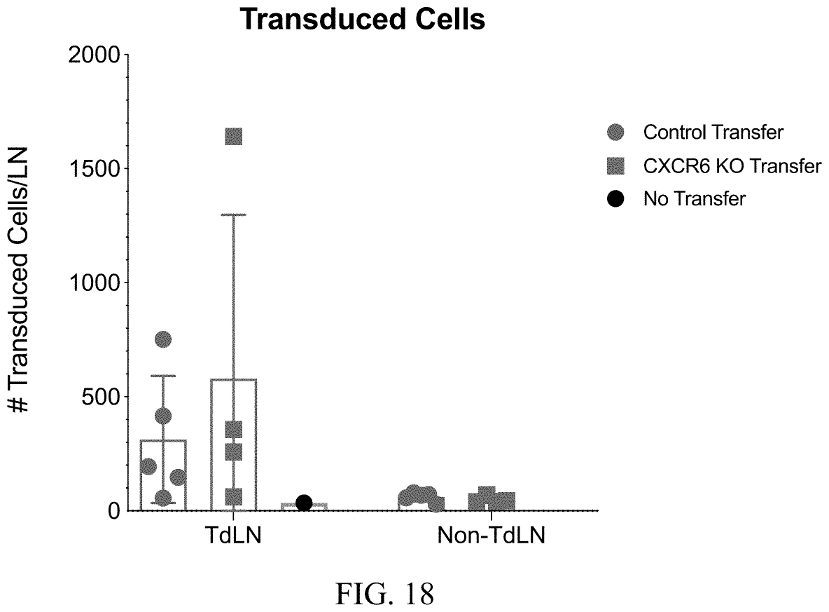

Applicants used the B16Ova/OTI T cell system and performed transfer experiments for no transfer control, transfer of control transduced cells, and transfer of CXCR6 CRISPR knockout cells (FIG. 11A,B). OTI are high-affinity transgenic CD8 T cells specific for Ova. Applicants observed that CXCR6 KO CD8+ T cells failed to control tumor growth. CXCR6 knockout did not affect total CD8+ T cell infiltration or OTI transferred T cell infiltration (FIG. 12). CXCR6 was efficiently deleted by CRISPR KO in vivo (FIG. 13A,B). CXCR6 knockout did not affect the PD1 or TIM3 populations or CD39 expression (FIG. 14A,B). Applicants observed that CXCR6 knockout may increase TCF-1 and decrease CX3CR1 (FIG. 15A,B). These cells are important for response to checkpoint blockade therapy (see, e.g., U.S. patent application Ser. No. 16/630,887). The Tcf7 gene encodes for TCF-1, a transcription factor (TF) involved in maintenance of stem-like memory precursor CD8⁺ TILS that are required for the success of checkpoint blockade therapy (Im et al., 2016; Kurtulus et al., 2019 Checkpoint Blockade Immunotherapy Induces Dynamic Changes in PD-1(−)CD8(+) Tumor-Infiltrating T Cells. Immunity 50, 181-194 e186; Miller et al., 2019 Subsets of exhausted CD8(+) T cells differentially mediate tumor control and respond to checkpoint blockade. Nat Immunol 20, 326-336; Siddiqui et al., 2019 Intratumoral Tcf1(+)PD-1(+)CD8(+) T Cells with Stem-like Properties Promote Tumor Control in Response to Vaccination and Checkpoint Blockade Immunotherapy. Immunity 50, 195-211 e110). Applicants observed that CXCR6 knockout did not change cytokine positive cells, but did show an increase of granzyme B+ T cells that do not degranulate (FIG. 16). Applicants observed that there was less effector differentiation in endogenous CD8+ T cells in mice receiving CXCR6-KO CD8+ T cells (PD1+ TIM3-CX3CR1+ and PD1+ TIM3+CX3CR1+) (FIG. 17). Applicants observed additional interesting trends in endogenous cells. For example, the lack of CXCR6 interactions affect myeloid populations which in turn interact with endogenous CD8 T cells less effectively. Thus, the lack of CXCR6 affects anti-tumor immunity in the entire tumor microenvironment. Applicants observed that there was similar T cell infiltration in the tumor-draining lymph node for control and CXCR6 KO cells indicating that the decreased anti-tumor immunity seen with the CXCR6 KO was not due to a decrease in infiltration (FIG. 18).

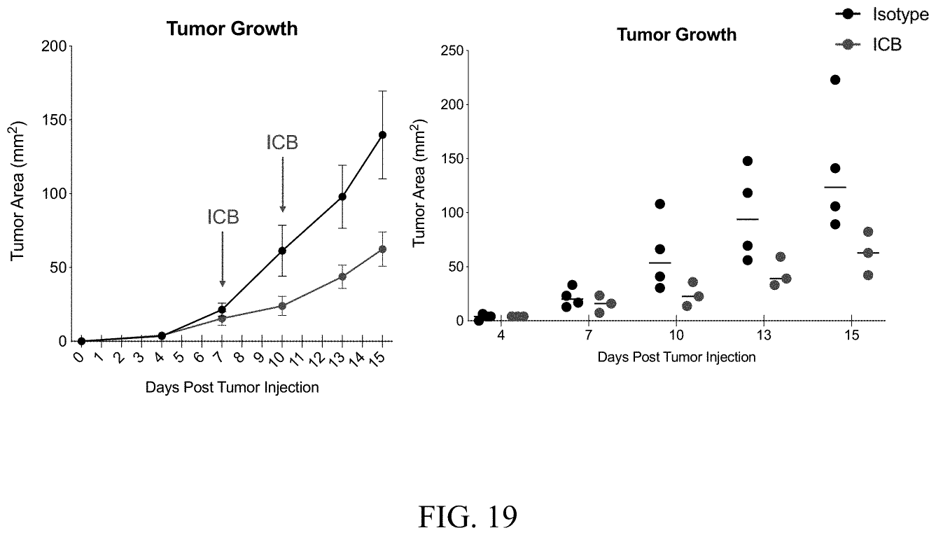

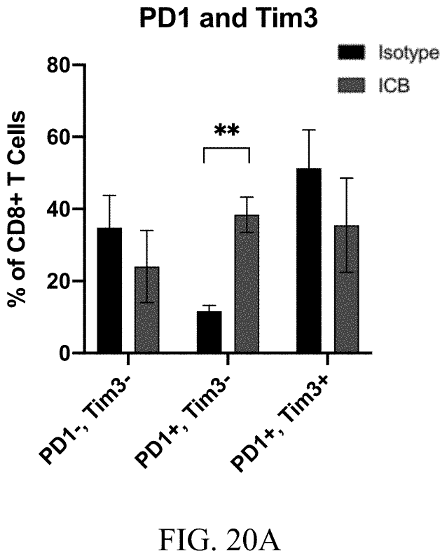

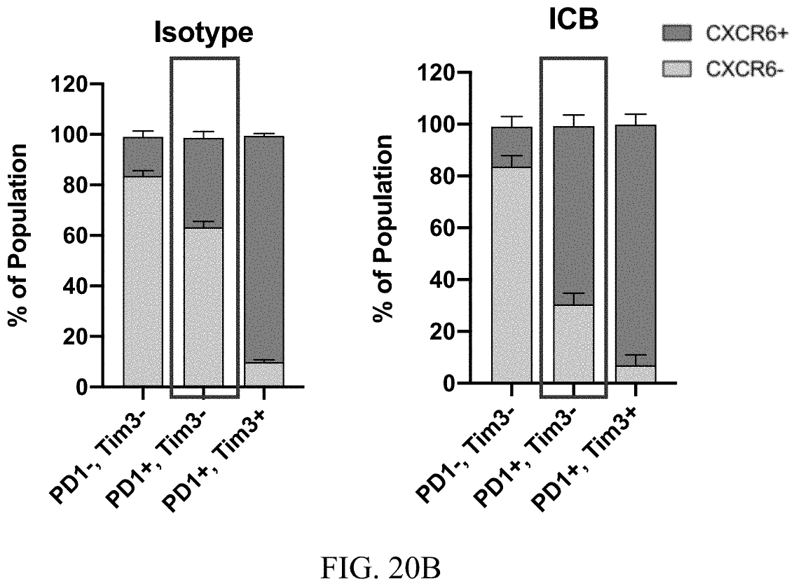

Applicants also investigated whether CXCR6 expression is altered after treatment with immune checkpoint blockade (ICB or CPB). Using wild type mice treated with ICB, Applicants observed decreased tumor growth as compared to isotype control (FIG. 19). Applicants observed that PD1+ Tim3− CD8+ T cells expanded upon ICB and had increased CXCR6 expression (FIG. 20A,B).

### Example 5—Discussion

The first key finding was that CXCR6 marks terminally dysfunctional or exhausted CD8+ TILs in multiple murine pre-clinical models of cancer and this aligned with the pan-cancer analyses of human data. The second key finding was that perturbation of CXCR6 with CRISPR/Cas9 decreases the ability of tumor-specific CD8+ T cells to control tumor growth. The third key finding was that administration of checkpoint blockade increases CXCR6 expression on CD8+ TILs. Thus, the data indicates that the CXCR6-CXCL16 interaction is important for preserving a level of functionality in tumor-specific CD8+ T cells and that without it, T cells become even more exhausted.

### REFERENCES

1. Sade-Feldman, M., Yizhak, K., Bjorgaard, S. L., Ray, J. P., de Boer, C. G., Jenkins, R. W., et al. (2018). Defining T Cell States Associated with Response to Checkpoint Immunotherapy in Melanoma. Cell, 175(4), 998-1013.e20. doi.org/10.1016/j.cell.2018.10.038
2. Sato, T., Thorlacius, H., Johnston, B., Staton, T. L., Xiang, W., Littman, D. R., & Butcher, E. C. (2004). Role for CXCR6 in Recruitment of Activated CD8+Lymphocytes to Inflamed Liver. The Journal of Immunology, 174(1), 277-283. doi.org/10.4049/jimmunol.174.1.277
3. Nanki, T., Shimaoka, T., Hayashida, K., Taniguchi, K., Yonehara, S., & Miyasaka, N. (2005). Pathogenic role of the CXCL16-CXCR6 pathway in rheumatoid arthritis. Arthritis & Rheumatism, 52(10), 3004-3014. doi.org/10.1002/art.21301
4. Yamauchi, R., Tanaka, M., Kume, N., Minami, M., Kawamoto, T., Togi, K., et al. (2004). Upregulation of SR-PSOX/CXCL16 and Recruitment of CD8+ T Cells in Cardiac Valves During Inflammatory Valvular Heart Disease. Arteriosclerosis, Thrombosis, and Vascular Biology, 24(2), 282-287. doi.org/10.1161/01.ATV.0000114565.42679.c6
5. Brinkman, E. K., Chen, T., Amendola, M., & van Steensel, B. (2014). Easy quantitative assessment of genome editing by sequence trace decomposition. Nucleic Acids Research, 42(22), e168-e168. doi.org/10.1093/nar/gku936
6. Sullender, M., Hegde, M., Vaimberg, E. W., Donovan, K. F., Smith, I., Tothova, Z., et al. (2016). Optimized sgRNA design to maximize activity and minimize off-target effects of CRISPR-Cas9. Nature Biotechnology, 34(2), 184-191. doi.org/10.1038/nbt.3437
7. Singer, M., Wang, C., Cong, Le., et al. (2016). A Distinct Gene Module for Dysfunction Uncoupled from Activation in Tumor-Infiltrating T Cells, Cell, 166(6), 1500-1511.e9. doi.org/10.1016/j.cell.2016.08.052
8. Heesch, K., Raczkowski, F., Schumacher, V., Hünemörder, S., Panzer, U., & Mittrücker, H.-W. (2014). The Function of the Chemokine Receptor CXCR6 in the T Cell Response of Mice against Listeria monocytogenes. Plos One, 9(5), e97701-9. doi.org/10.1371/journal.pone.0097701
9. Kumar, B. V., Ma, W., Miron, M., Granot, T., Guyer, R. S., Carpenter, D. J., et al. (2017). Human Tissue-Resident Memory T Cells Are Defined by Core Transcriptional and Functional Signatures in Lymphoid and Mucosal Sites. CellReports, 20(12), 2921-2934. doi.org/10.1016/j.celrep.2017.08.078

The invention is further described in the following numbered paragraphs:

1. A population of CD8+ T cells comprising one or more CD8+ T cells modified ex vivo to comprise altered expression, activity and/or function of:
a. one or more genes selected from the group consisting of CXCR6, NDFIP2, CD82, LSP1, FKBP1A, PKM, ACP5, PHLDA1, AKAP5, NAB1, SIRPG, DUSP4, RGS1, GAPDH, RBPJ, TNFRSF9, MIR155HG, CD27, CD2, TNFSF4, CXCL13, SAMSN1, EPSTI1, SARDH, CD74, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1; or
b. one or more genes selected from the group consisting of CD82, PKM, ACP5, AKAP5, NAB1, SIRPG, RGS1, TNFRSF9, MIR155HG, CD27, CD2, TNFSF4, CXCL13, SAMSN1, EPSTI1, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1; or
c. one or more genes selected from the group consisting of CD82, PKM, ACP5, AKAP5, NAB1, SIRPG, RGS1, TNFRSF9, MIR155HG, CD27, CD2, TNFSF4, CXCL13, SAMSN1, EPSTI1, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1, and one or more genes selected from the group consisting of NDFIP2, LSP1, CXCR6, FKBP1A, PHLDA1, DUSP4, GAPDH, RBPJ, SARDH and CD74; or
d. one or more genes selected from the group consisting of RBPJ, NAB1, TOX, IFI6, ZBED2, IFI16, CCND2, PHLDA1 and ETV1; or
e. one or more genes selected from the group consisting of CXCR6, TNFRSF9, SIRPG, CD27, CD2, TNFSF4, HLA-DRA, CD8A, HLA-DRB1, HLA-DMA, HLA-DPA1, CD74, TRAF5, BST2, VCAM1, ITGAE, CLEC2D, CD38, ANXA5, CD82, HLA-A, TNFRSF1B, FASLG, PAG1, RAB27A, LY6E, IGFLR1, CD3D and HLA-DRB5; or
f. one or more genes selected from the group consisting of ACP5, CXCL13, FAM3C and ISG15.
2. The population of CD8+ T cells of paragraph 1, wherein the one or more CD8+ T cells are modified to increase expression, activity and/or function of CXCR6.
3. The population of CD8+ T cells of paragraph 2, wherein a nucleotide sequence encoding for CXCR6 is introduced to the one or more CD8+ T cells ex vivo.
4. The population of CD8+ T cells of paragraph 2, wherein a sequence specific genome editing system is introduced ex vivo to activate or enhance expression of endogenous CXCR6.
5. An enriched population of CD8+ T cells obtained by enriching for CXCR6+ CD8+ T cells from an ex vivo population of immune cells.
6. The enriched population of CD8+ T cells of paragraph 5, wherein the T cells are further enriched for PD1+ TIM3− CD8+ T cells, whereby the population of cells is enriched for CXCR6+ PD1+ TIM3− CD8+ T cells.
7. The enriched population of CD8+ T cells of paragraph 5 or 6, wherein the T cells are enriched using antibodies specific to CXCR6, PD1, TIM3 and/or CD8.
8. The population of CD8+ T cells of any one of paragraphs 1 to 7, wherein the CD8+ T cells are further modified to comprise decreased expression, activity and/or function of one or more genes selected from the group consisting of HAVCR2, PDCD1, TIGIT, CTLA4, LAG3 and ENTPD1.
9. The population of CD8+ T cells of any one of paragraphs 1 to 8, wherein the CD8+ T cells are modified to temporarily decrease expression, activity and/or function of the one or more genes.
10. The population of CD8+ T cells of any one of paragraphs 1 to 9, wherein the CD8+ T cells express a CRISPR system.
11. The population of CD8+ T cells of paragraph 10, wherein the CRISPR system comprises a CRISPR base editing system, a prime editor system, or a CAST system.
12. The population of CD8+ T cells of any one of paragraphs 1 to 11, wherein the population of CD8+ T cells comprises CD8+ T cells expanded ex vivo.
13. The population of CD8+ T cells of any one of paragraphs 1 to 12, wherein the CD8+ T cells are tumor infiltrating lymphocytes (TILs).
14. The population of CD8+ T cells of any one of paragraphs 1 to 13, wherein the CD8+ T cells are specific for a tumor antigen.
15. The population of CD8+ T cells of any one of paragraphs 1 to 14, wherein the CD8+ T cells are modified to express an exogenous T cell receptor (TCR) or chimeric antigen receptor (CAR).
16. The population of CD8+ T cells of any one of paragraphs 1 to 15, wherein the CD8+ T cells express a suicide switch gene.
17. The population of CD8+ T cells of any one of paragraphs 1 to 16, wherein the CD8+ T cells are autologous cells obtained from a subject suffering from cancer.
18. The population of CD8+ T cells of any one of paragraphs 1 to 16, wherein the CD8+ T cells are allogenic cells further modified to prevent transplant rejection.
19. A pharmaceutical composition comprising the population of cells according to any one of paragraphs 1 to 18.
20. A method of treating cancer comprising administering the pharmaceutical composition of paragraph 19 to a subject in need thereof.
21. A method of treating cancer comprising administering to a subject in need thereof one or more agents capable of modulating expression, activity, and/or function of:
a. one or more genes selected from the group consisting of CXCR6, NDFIP2, CD82, LSP1, FKBP1A, PKM, ACP5, PHLDA1, AKAP5, NAB1, SIRPG, DUSP4, RGS1, GAPDH, RBPJ, TNFRSF9, MIR155HG, CD27, CD2, TNFSF4, CXCL13, SAMSN1, EPSTI1, SARDH, CD74, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1; or
b. one or more genes selected from the group consisting of CD82, PKM, ACP5, AKAP5, NAB1, SIRPG, RGS1, TNFRSF9, MIR155HG, CD27, CD2, TNFSF4, CXCL13, SAMSN1, EPSTI1, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1; or
c. one or more genes selected from the group consisting of CD82, PKM, ACP5, AKAP5, NAB1, SIRPG, RGS1, TNFRSF9, MIR155HG, CD27, CD2, TNFSF4, CXCL13, SAMSN1, EPSTI1, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1, and one or more genes selected from the group consisting of NDFIP2, LSP1, CXCR6, FKBP1A, PHLDA1, DUSP4, GAPDH, RBPJ, SARDH and CD74; or
d. one or more genes selected from the group consisting of RBPJ, NAB1, TOX, IFI6, ZBED2, IFI16, CCND2, PHLDA1 and ETV1; or
e. one or more genes selected from the group consisting of CXCR6, TNFRSF9, SIRPG, CD27, CD2, TNFSF4, HLA-DRA, CD8A, HLA-DRB1, HLA-DMA, HLA-DPA1, CD74, TRAF5, BST2, VCAM1, ITGAE, CLEC2D, CD38, ANXA5, CD82, HLA-A, TNFRSF1B, FASLG, PAG1, RAB27A, LY6E, IGFLR1, CD3D and HLA-DRB5; or
f. one or more genes selected from the group consisting of ACP5, CXCL13, FAM3C and ISG15.
22. The method of paragraph 21, wherein CXCR6 expression, activity, and/or function is enhanced.
23. The method of paragraph 22, wherein CXCL16 expression, activity, and/or function is enhanced.
24. The method of paragraph 21, wherein CXCR6 expression, activity, and/or function is reduced.
25. The method of paragraph 24, wherein one or more agents capable of reducing expression, activity, and/or function of CXCR6 is administered in combination with anti-PD-1, anti-CTLA4, anti-PD-L1, anti-TIM3, anti-TIGIT, anti-LAG3, or combination thereof.
26. The method of any one of paragraphs 21 to 25, further comprising administering one or more agents capable of decreasing expression, activity, and/or function of one or more genes selected from the group consisting of HAVCR2, PDCD1, TIGIT, CTLA4, LAG3, ENTPD1 and PD-L1.
27. The method of paragraph 21, wherein the one or more agents target a ligand, receptor or substrate of the one or more genes.
28. The method of any one of paragraphs 21 to 27, wherein the one or more agents comprise an antibody, antibody-like protein scaffold, aptamer, small molecule, genetic modifying agent, protein, nucleic acid or any combination thereof.
29. The method of paragraph 28, wherein the one or more agents comprise one or more antibodies targeting one or more cell surface proteins or one or more ligands/receptors of the one or more cell surface proteins, wherein the one or more cell surface proteins are selected from the group consisting of CXCR6, TNFRSF9, SIRPG, CD27, CD2, TNFSF4, HLA-DRA, CD8A, HLA-DRB1, HLA-DMA, HLA-DPA1, CD74, TRAF5, BST2, VCAM1, ITGAE, CLEC2D, CD38, ANXA5, CD82, HLA-A, TNFRSF1B, FASLG, PAG1, RAB27A, LY6E, IGFLR1, CD3D and HLA-DRB5.
30. The method of paragraph 29, wherein the surface protein is CXCR6 and the ligand targeted is CXCL16.
31. The method of paragraph 28, wherein the one or more agents comprise one or more antibodies targeting one or more secreted proteins or one or more receptors of the one or more secreted proteins, wherein the one or more secreted proteins are selected from the group consisting of ACP5, CXCL13, FAM3C and ISG15.
32. The method of paragraph 28, wherein the one or more agents comprise one or more antibodies targeting one or more genes selected from the group consisting of HAVCR2, PDCD1, TIGIT, CTLA4, LAG3, ENTPD1 and PD-L1.
33. The method of paragraph 32, wherein the one or more antibodies is selected from the group consisting of Ipilimumab, Nivolumab, Pembrolizumab and Atezolizumab.
34. The method of paragraph 28, wherein the one or more agents comprise an inhibitor of ENTPD1.
35. The method of paragraph 34, wherein the inhibitor is selected from the group consisting of 6-N,N-Diethyl-d-β-γ-dibromomethylene adenosine triphosphate (ARL 67156), 8-thiobutyladenosine 5′-triphosphate (8-Bu-S-ATP), polyoxymetate-1 (POM-1) and α,β-methylene ADP (APCP).
36. The method of paragraph 28, wherein the small molecule is a small molecule degrader.
37. The method of paragraph 28, wherein the genetic modifying agent comprises a CRISPR system, RNAi system, a zinc finger nuclease system, a TALE system, or a meganuclease.
38. The method of paragraph 37, wherein the CRISPR system comprises a CRISPR base editing system, a prime editor system, or a CAST system.
39. A method of detecting dysfunctional T cells comprising detecting a dysfunctional gene signature in T cells obtained from a subject in need thereof, wherein the dysfunctional gene signature comprises expression of:
a. one or more genes selected from the group consisting of CXCR6, NDFIP2, CD82, LSP1, FKBP1A, PKM, ACP5, PHLDA1, AKAP5, NAB1, SIRPG, DUSP4, RGS1, GAPDH, RBPJ, TNFRSF9, MIR155HG, CD27, CD2, TNFSF4, CXCL13, SAMSN1, EPSTI1, SARDH, CD74, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1; or
b. one or more genes selected from the group consisting of CD82, PKM, ACP5, AKAP5, NAB1, SIRPG, RGS1, TNFRSF9, MIR155HG, CD27, CD2, TNFSF4, CXCL13, SAMSN1, EPSTI1, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1; or
c. one or more genes selected from the group consisting of CD82, PKM, ACP5, AKAP5, NAB1, SIRPG, RGS1, TNFRSF9, MIR155HG, CD27, CD2, TNFSF4, CXCL13, SAMSN1, EPSTI1, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1, and one or more genes selected from the group consisting of NDFIP2, LSP1, CXCR6, FKBP1A, PHLDA1, DUSP4, GAPDH, RBPJ, SARDH and CD74; or
d. one or more genes selected from the group consisting of RBPJ, NAB1, TOX, IFI6, ZBED2, IFI16, CCND2, PHLDA1 and ETV1; or
e. one or more genes selected from the group consisting of CXCR6, TNFRSF9, SIRPG, CD27, CD2, TNFSF4, HLA-DRA, CD8A, HLA-DRB1, HLA-DMA, HLA-DPA1, CD74, TRAF5, BST2, VCAM1, ITGAE, CLEC2D, CD38, ANXA5, CD82, HLA-A, TNFRSF1B, FASLG, PAG1, RAB27A, LY6E, IGFLR1, CD3D and HLA-DRB5; or
f. one or more genes selected from the group consisting of ACP5, CXCL13, FAM3C and ISG15.
40. The method of paragraph 39, wherein the dysfunctional gene signature further comprises expression of one or more genes selected from the group consisting of HAVCR2, PDCD1, TIGIT, CTLA4, LAG3 and ENTPD1.
41. The method of paragraph 39 or 40, wherein the T cells are sorted by FACS.
42. The method of any one of paragraphs 39 to 41, wherein the T cells are detected using RNA sequencing.
43. The method of paragraph 42, wherein the T cells are detected using single cell RNA sequencing.
44. The method of paragraph 39 or 40, wherein the T cells are detected using immunohistochemistry.
45. The method of any one of paragraphs 39 to 44, further comprising determining if the subject is responsive to checkpoint blockade (CPB) monotherapy, wherein detecting the dysfunctional gene signature in a subject indicates that the subject is not responsive to checkpoint blockade (CPB) monotherapy.
46. The method of paragraph 45, wherein the subject that is not responsive has a higher proportion of T cells expressing the dysfunctional signature as compared to T cells not expressing the dysfunctional signature.
47. The method of paragraph 45 or 46, further comprising:
treating a subject not having a dysfunctional gene signature with checkpoint blockade (CPB) monotherapy; or
treating a subject having a dysfunctional signature according to any of paragraphs 20 to 38; or
treating a subject having a dysfunctional signature with one or more treatments selected from the group consisting of surgery, targeted therapy, chemotherapy and radiation therapy; and, optionally, immunotherapy.
48. The method of any one of paragraphs 39 to 44, wherein the method is for monitoring checkpoint blockade (CPB) therapy in a subject in need thereof, wherein the CPB therapy is effective if CXCR6 expression increases in CD8+ T cells in the subject.
49. A method of screening for T cell modulating agents, comprising:
a. treating a population of T cells having a dysfunctional gene signature according to paragraph 39 or 40 with a test agent; and
b. detecting a decrease in the dysfunctional gene signature as compared to an untreated population of T cells.
50. A kit comprising reagents to detect at least one gene according to the gene signature as defined in paragraphs 39 or 40.
51. A method of identifying a pan-tumor signature comprising:
a. applying dimensionality reduction on two or more single cell RNA sequencing cohorts comprising dysfunctional T cells simultaneously;
b. identifying genes that characterize both dysfunctional CD8 T cells and regulatory (CD4) T cells; and
c. using RNA velocity to identify genes that are expressed early and/or late during exhaustion.
52. The method of paragraph 51, wherein dimensionality reduction comprises mixed-NW.
53. A bispecific antibody capable of enhancing interaction between dendritic cells (DCs) and PD1+ CD8+ T cells, wherein the bispecific antibody binds to a surface protein on the T cells and a DC surface protein.
54. The bispecific antibody of paragraph 53, wherein the T cell surface protein is selected from the group consisting of CXCR6 and PD1.
55. The bispecific antibody of paragraph 53, wherein the DC surface protein is selected from the group consisting of CXCL16, CD11c, XCR1 and CD103.
56. A method of treating cancer comprising administering to a subject in need thereof the bispecific antibody according to any of paragraphs 53 to 55.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A population of CD8+ T cells modulated ex vivo to increase expression, activity and/or function of CXCR6.

2. The population of CD8+ T cells of claim 1, wherein a nucleotide sequence encoding for CXCR6 is introduced to the one or more CD8+ T cells ex vivo; or
wherein a sequence specific genome editing system is introduced ex vivo to activate or enhance expression of endogenous CXCR6.

3. (canceled)

4. The population of CD8+ T cells of claim 1, wherein the population is obtained by enriching for CXCR6+ CD8+ T cells from an ex vivo population of immune cells preferably,
wherein the T cells are further enriched for PD1+ TIM3− CD8+ T cells, whereby the population of cells is enriched for CXCR6+ PD1+ CD8+ T cells; and/or
wherein the T cells are enriched using antibodies specific to CXCR6, PD1, TIM3 and/or CD8.

5-6. (canceled)

7. The population of CD8+ T cells of claim 1, wherein the CD8+ T cells are further modified to comprise decreased expression, activity and/or function of one or more genes selected from the group consisting of HAVCR2, PDCD1, TIGIT, CTLA4, LAG3 and ENTPD1; and/or
wherein the CD8+ T cells are tumor infiltrating lymphocytes (TILS); and/or
wherein the CD8+ T cells are specific for a tumor antigen; and/or
wherein the CD8+ T cells are modified to express an exogenous T cell receptor (TCR) or chimeric antigen receptor (CAR); and/or
wherein the CD8+ T cells express a suicide switch gene.

8-11. (canceled)

12. The population of CD8+ T cells of claim 1, wherein the CD8+ T cells are autologous cells obtained from a subject suffering from cancer; or wherein the CD8+ T cells are allogenic cells further modulated to prevent transplant rejection.

13. (canceled)

14. A pharmaceutical composition comprising the population of cells according to claim 1.

15. A method of treating cancer comprising administering the pharmaceutical composition of claim 14 to a subject in need thereof.

16. A method of treating cancer comprising administering to a subject in need thereof one or more agents capable of modulating expression, activity, and/or function of CXCR6, preferably,
wherein CXCR6 expression, activity, and/or function in T cells is enhanced; or
wherein CXCL16 expression, activity, and/or function is enhanced; or
wherein CXCR6 expression, activity, and/or function is reduced, preferably, wherein one or more agents capable of reducing expression, activity, and/or function of CXCR6 is administered in combination with anti-PD-1, anti-CTLA4, anti-PD-L1, anti-TIM3, anti-TIGIT, anti-LAG3, or combination thereof.

17-20. (canceled)

21. The method of claim 16, further comprising administering one or more agents capable of decreasing expression, activity, and/or function of one or more genes selected from the group consisting of HAVCR2, PDCD1, TIGIT, CTLA4, LAG3, ENTPD1 and PD-L1.

22. The method of claim 16, wherein the one or more agents comprise an antibody, antibody-like protein scaffold, aptamer, small molecule, genetic modifying agent, CXCL16 protein or fragment, nucleic acid or any combination thereof.

23. The method of claim 22, wherein the one or more agents comprise one or more antibodies targeting CXCR6; and/or
wherein CXCL16 is targeted by the one or more agents; and/or
wherein the one or more agents comprise one or more antibodies targeting one or more genes selected from the group consisting of HAVCR2, PDCD1, TIGIT, CTLA4, LAG3, ENTPD1 and PD-L1, preferably, wherein the one or more antibodies is selected from the group consisting of Ipilimumab, Nivolumab, Pembrolizumab and Atezolizumab; and/or
wherein the one or more agents comprise an inhibitor of ENTPD1, preferably, wherein the inhibitor is selected from the group consisting of 6-N,N-Diethyl-d-β-γ-dibromomethylene adenosine triphosphate (ARL 67156), 8-thiobutyladenosine 5′-triphosphate (8-Bu-S-ATP), polyoxymetate-1 (POM-1) and α,β-methylene ADP (APCP); and/or
wherein the small molecule is a small molecule degrader; and/or
wherein the genetic modifying agent comprises a CRISPR system, RNAi system, a zinc finger nuclease system, a TALE system, or a meganuclease designed to target the CXCR6 gene, target negative regulators of CXCR6, modify chromatin surrounding the CXCR6 gene, target the promoter or enhancers regulating the CXCR6 gene, or substitute the CXCR6 gene with an enhanced expression cassette.

24-30. (canceled)

31. A method of detecting dysfunctional T cells comprising detecting a dysfunctional gene signature in T cells obtained from a subject in need thereof, wherein the dysfunctional gene signature comprises expression of:
a. one or more genes selected from the group consisting of CXCR6, NDFIP2, CD82, LSP1, FKBP1A, PKM, ACP5, PHLDA1, AKAP5, NAB1, SIRPG, DUSP4, RGS1, GAPDH, RBPJ, TNFRSF9, MIR155HG, CD27, CD2, TNFSF4, CXCL13, SAMSN1, EPSTI1, SARDH, CD74, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1; or
b. one or more genes selected from the group consisting of CD82, PKM, ACP5, AKAP5, NAB1, SIRPG, RGS1, TNFRSF9, MIR155HG, CD27, CD2, TNFSF4, CXCL13, SAMSN1, EPSTI1, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1; or
c. one or more genes selected from the group consisting of CD82, PKM, ACP5, AKAP5, NAB1, SIRPG, RGS1, TNFRSF9, MIR155HG, CD27, CD2, TNFSF4, CXCL13, SAMSN1, EPSTI1, APOBEC3C, HLA-DRA, CD8A, HLA-DRB1, TNS3, FUT8, HLA-DMA, TOX, GOLIM4, IFI6, LYST, HLA-DPA1, FAM3C, ZBED2, PAG1, TRAF5, RAB27A, BST2, CLEC2D, CD38, LY6E, VCAM1, ITGAE, ISG15, XAF1, ANXA5, IFI16, RHOA, HLA-A, LINC00158, CCND2, TNFRSF1B, SHFM1, GBP5, TNIP3, TYMP, PLSCR1, MX1, GBP2, UBC, FASLG, SNAP47, GALM, IGFLR1, SH2D2A, MYO7A, CD3D, AFAP1L2, HLA-DRB5, FABP5, HMOX1 and ETV1, and one or more genes selected from the group consisting of NDFIP2, LSP1, CXCR6, FKBP1A, PHLDA1, DUSP4, GAPDH, RBPJ, SARDH and CD74; or
d. one or more genes selected from the group consisting of RBPJ, NAB1, TOX, IFI6, ZBED2, IFI16, CCND2, PHLDA1 and ETV1; or
e. one or more genes selected from the group consisting of CXCR6, TNFRSF9, SIRPG, CD27, CD2, TNFSF4, HLA-DRA, CD8A, HLA-DRB1, HLA-DMA, HLA-DPA1, CD74, TRAF5, BST2, VCAM1, ITGAE, CLEC2D, CD38, ANXA5, CD82, HLA-A, TNFRSF1B, FASLG, PAG1, RAB27A, LY6E, IGFLR1, CD3D and HLA-DRB5; or
f. one or more genes selected from the group consisting of ACP5, CXCL13, FAM3C and ISG15,
preferably, wherein the dysfunctional gene signature further comprises expression of one or more genes selected from the group consisting of HAVCR2, PDCD1, TIGIT, CTLA4, LAG3 and ENTPD1.

32. (canceled)

33. The method of claim 31, further comprising determining if the subject is responsive to checkpoint blockade (CPB) monotherapy, wherein detecting the dysfunctional gene signature in a subject indicates that the subject is not responsive to checkpoint blockade (CPB) monotherapy, preferably, wherein the subject that is not responsive has a higher proportion of T cells expressing the dysfunctional signature as compared to T cells not expressing the dysfunctional signature.

34. (canceled)

35. The method of claim 31, further comprising:
treating a subject not having a dysfunctional gene signature with checkpoint blockade (CPB) monotherapy; or
treating a subject having a dysfunctional signature according to claim 15; or
treating a subject having a dysfunctional signature with one or more treatments selected from the group consisting of surgery, targeted therapy, chemotherapy and radiation therapy; and, optionally, immunotherapy.

36. The method of claim 31, wherein the method is for monitoring checkpoint blockade (CPB) therapy in a subject in need thereof, wherein the CPB therapy is effective if CXCR6 expression increases in CD8+ T cells in the subject.

37. A method of screening for T cell modulating agents, comprising:
a. treating a population of T cells having a dysfunctional gene signature according to claim 31 with a test agent; and
b. detecting a decrease in the dysfunctional gene signature as compared to an untreated population of T cells.

38. A kit comprising reagents to detect at least one gene according to the gene signature as defined in claim 31.

39. A method of identifying a pan-tumor signature comprising:
a. applying dimensionality reduction on two or more single cell RNA sequencing cohorts comprising dysfunctional T cells simultaneously, preferably, wherein dimensionality reduction comprises mixed-NMF;
b. identifying genes that characterize both dysfunctional CD8 T cells and regulatory (CD4) T cells; and
c. using RNA velocity to identify genes that are expressed early and/or late during exhaustion.

40. (canceled)

41. A bispecific antibody capable of enhancing interaction between dendritic cells (DCs) and PD1+ CD8+ T cells, wherein the bispecific antibody binds to a surface protein on the T cells and a DC surface protein, preferably,
wherein the T cell surface protein is selected from the group consisting of CXCR6 and PD1; and/or
wherein the DC surface protein is selected from the group consisting of CXCL16, CD11c, XCR1 and CD103.

42-43. (canceled)

44. A method of treating cancer comprising administering to a subject in need thereof the bispecific antibody according to claim 41.


SEQUENCE LISTING (23 sequences)

SEQ ID NO: 1 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 9 aa, Homo sapiens
IEVMYPPPY

SEQ ID NO: 3 - protein, 288 aa, Artificial Sequence
  Synthetic N-terminal capping region
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 4 - protein, 183 aa, Artificial Sequence
  Synthetic C-terminal capping region
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 5 - protein, 7 aa, Simian virus 40
PKKKRKV

SEQ ID NO: 6 - protein, 10 aa, Simian virus 40
PKKKRKVEAS

SEQ ID NO: 7 - protein, 16 aa, Artificial Sequence
  Synthetic nucleoplasmin bipartite NLS
KRPAATKKAGQAKKKK

SEQ ID NO: 8 - protein, 9 aa, Homo sapiens
PAAKRVKLD

SEQ ID NO: 9 - protein, 11 aa, Homo sapiens
RQRRNELKRSP

SEQ ID NO: 10 - protein, 38 aa, Homo sapiens
NQSSNFGPMKGGNFGGRSSGPYGGGGQYFAKPRNQGGY

SEQ ID NO: 11 - protein, 42 aa, Homo sapiens
RMRIZFKNKGKDTAELRRRRVEVSVELRKAKKDEQILKRRNV

SEQ ID NO: 12 - protein, 8 aa, Homo sapiens
VSRKRPRP

SEQ ID NO: 13 - protein, 8 aa, Homo sapiens
PPKKARED

SEQ ID NO: 14 - protein, 8 aa, Homo sapiens
PQPKKKPL

SEQ ID NO: 15 - protein, 12 aa, Mus sp.
SALIKKKKKMAP

SEQ ID NO: 16 - protein, 5 aa, Influenza virus
DRLRR

SEQ ID NO: 17 - protein, 7 aa, Influenza virus
PKQKKRK

SEQ ID NO: 18 - protein, 10 aa, Hepatitis D virus
RKLKKKIKKL

SEQ ID NO: 19 - protein, 10 aa, Mus sp.
REKKKFLKRR

SEQ ID NO: 20 - protein, 20 aa, Homo sapiens
KRKGDEVDGVDEVAKKKSKK

SEQ ID NO: 21 - protein, 17 aa, Homo sapiens
RKCLQAGMNLEARKTKK

SEQ ID NO: 22 - DNA, 45 bp, Artificial Sequence
  Synthetic
gctgacctggtgtttgtctgtactctgcccttttgggcctatgca

SEQ ID NO: 23 - DNA, 45 bp, Artificial Sequence
  Synthetic
cgactggaccacaaacagacatgagacgggaaaacccggatacgt